(19) United States
(12) Patent Application Publication
(10) Pub. No.: US 2024/0120028 A1
(43) Pub. Date: Apr. 11, 2024

(54) Learning Architecture and Pipelines for Granular Determination of Genetic Ancestry

(71) Applicant: 23andMe, Inc., Sunnyvale, CA (US)

(72) Inventors: Timothy B. Do, Mountain View, CA (US); Nathaniel McQuay, San Jose, CA (US); Rachel E. Lopatin, Santa Clara, CA (US); Manoj Ganesan, Sunnyvale, CA (US); Subarnarekha Sinha, Los Altos, CA (US); Andrew C. Seaman, San Jose, CA (US); William A. Freyman, Oakland, CA (US); Katarzyna Bryc, Arlington, MA (US); Steven J. Micheletti, San Bruno, CA (US); Peter R. Wilton, Duluth, MN (US); Samantha G. Ancona Esselmann, San Francisco, CA (US)

(21) Appl. No.: 18/374,265

(22) Filed: Sep. 28, 2023

Related U.S. Application Data
(60) Provisional application No. 63/413,276, filed on Oct. 5, 2022.

(51) Int. Cl. G16B 20/40; G06N 20/20; G16B 40/20; G16B 45/00

(52) U.S. Cl. CPC G16B 20/40; G06N 20/20; G16B 40/20; G16B 45/00

(57) ABSTRACT

An example embodiment may involve estimating, from genetic data of a plurality of individuals, identity-by-descent (IBD) segments; forming, from the IBD segments, a relationship graph representing genetic linkages between the individuals; determining, by applying a stochastic block model to the relationship graph, a plurality of genetic groups, wherein each of the genetic groups is assigned a respective subset of the individuals who share a greater amount of IBD segment length with one another than with a further respective subset of the individuals who are in other of the genetic groups; and training, for each of the genetic groups, a respective classifier based on (i) input including genome-wide local ancestry proportions of the individuals and sums of IBD segments for the individuals in the respective genetic group, and (ii) associated output of assignments of the individuals to the genetic groups.

### CROSS-REFERENCE TO RELATED APPLICATION

This application claims priority to U.S. provisional patent application No. 63/413,276, filed Oct. 5, 2022, which is hereby incorporated by reference in its entirety.

### BACKGROUND

Identity-by-descent (IBD) is a term used to refer to matching segments of deoxyribonucleic acid (DNA) shared by two or more individuals, where these individuals inherited the segments from a common ancestor without recombination. IBD segments match when the alleles on a paternal or maternal chromosome are identical to one another above some pre-determined threshold. The length of IBD segments can vary based on the number of generations between the individuals and the ancestor (e.g., longer segments tend to be found when the common ancestor is more recent than when the common ancestor is not as recent).

Many computer-implemented genetic analysis techniques estimate the length and genomic location of IBD segments in order to identify genetic relatives, inherited traits, and health-related predispositions. But genetic datasets are quite large, often including at least partial genomes of millions of individuals. Consequently, improving the speed and accuracy of IBD determinations can be valuable in the fields of genetics and medicine, and in other fields as well.

### SUMMARY

The embodiments herein involve machine learning and/or related techniques that can be used to rapidly discover highly-granular genetic ancestry relationships between individuals and groups. These techniques streamline and automate the complex process of detecting fine-scale genetic group structure, as well as developing and deploying models (e.g., in the form of classifiers) that can assign individuals to genetic groups. Thus, these embodiments are a technological and scientific advance in terms of providing new approaches for genetic group determination. Moreover, another advance described herein is the custom computing infrastructure used to systematize the delivery of new genetic ancestry results.

Some embodiments may employ probabilistic IBD clustering using stochastic block models (SBMs) to identify genetic group structure. These embodiments may also train a plurality of logistic regression classifiers to provide assignments of individuals to the genetic groups (e.g., one classifier per group). Prior approaches were unable to determine genetic group structure from genetic datasets in a single pipeline. Further, the use of SBMs increases the accuracy of such determinations.

The determined genetic groups can be associated with geographic regions that are not limited to being defined by political or administrative boundaries. This, along with other possible textual, image, audio, and/or video content can be provided to end users who are reviewing genetic information related to a genetic group or an individual belonging to such a group.

Accordingly, a first example embodiment may involve estimating, from genetic data of a plurality of individuals, IBD segments; forming, from the IBD segments, a relationship graph representing genetic linkages between the individuals; determining, by applying a stochastic block model to the relationship graph, a plurality of genetic groups, wherein each of the genetic groups is assigned a respective subset of the individuals who share a greater amount of IBD segment length with one another than with a further respective subset of the individuals who are in other of the genetic groups; and training, for each of the genetic groups, a respective classifier based on (i) input including genome-wide local ancestry proportions of the individuals and sums of IBD segments for all of the individuals in the respective genetic group, and (ii) associated output of assignments of the individuals to the genetic groups.

A second example embodiment may involve receiving particular genetic data of a particular individual; determining, from the particular genetic data of the particular individual, a particular genome-wide local ancestry proportion of the particular individual; applying each of a plurality of classifiers respectively associated with genetic groups to: the particular genome-wide local ancestry proportion of the particular individual and sums of IBD segments for all individuals in the associated genetic group, wherein the classifiers were respectively trained based on: (i) input including genome-wide local ancestry proportions of the individuals and sums of IBD segments for the individuals in the associated genetic group, and (ii) associated output of assignments of the individuals to the genetic groups; and based on results of applying each of the plurality of classifiers, assigning the particular individual to at least one of the genetic groups.

A third example embodiment may involve receiving, on behalf of a particular individual of a plurality of individuals, a deletion request, wherein one or more databases configured to store (i) genetic data for the plurality of individuals, and (ii) a plurality of classifiers respectively associated with genetic groups, wherein each of the classifiers was trained with the genetic data and is deployed to be used in production to predict whether further individuals belong to its associated genetic group; in response to receiving the deletion request: (i) deleting, from the genetic data, particular genetic data of the particular individual, (ii) determining a set of one or more of the classifiers that were trained using at least some of the particular genetic data, and (iii) retraining the set of one or more of the classifiers with the genetic data after deletion; and redeploying the set of one or more of the classifiers as retrained.

In a fourth example embodiment, an article of manufacture may include a non-transitory computer-readable medium, having stored thereon program instructions that, upon execution by a computing system, cause the computing system to perform operations in accordance with the first, second, and/or third example embodiment.

In a fifth example embodiment, a computing system may include at least one processor, as well as memory and program instructions. The program instructions may be stored in the memory, and upon execution by the at least one processor, cause the computing system to perform operations in accordance with the first, second, and/or third example embodiment.

In a sixth example embodiment, a system may include various means for carrying out each of the operations of the first, second, and/or third example embodiment.

These, as well as other embodiments, aspects, advantages, and alternatives, will become apparent to those of ordinary skill in the art by reading the following detailed description, with reference where appropriate to the accompanying drawings. Further, this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE DRAWINGS

FIG. 1 illustrates a schematic drawing of a computing device, in accordance with example embodiments.

FIG. 2 illustrates a schematic drawing of a server device cluster, in accordance with example embodiments.

FIG. 3 is a map depicting course-grained genetic groups associated with political and administrative regions of a country, in accordance with example embodiments.

FIG. 4 depicts the main steps of a process for determining fine-grained genetic groups associated with geographic regions, in accordance with example embodiments.

FIG. 5 depicts data used by an algorithm for fast IBD estimates that are robust to genotype and phasing errors, in accordance with example embodiments.

FIG. 6 depicts a clustering of individuals into genetic groups based on their IBD segments, in accordance with example embodiments.

FIG. 7 depicts training classifiers for genetic groups based on the clustering, in accordance with example embodiments.

FIG. 8 depicts a map based on genetic groups, in accordance with example embodiments.

FIG. 9A depicts a model development architecture, in accordance with example embodiments.

FIG. 9B depicts a training pipeline, in accordance with example embodiments.

FIG. 9C depicts an impact analysis pipeline, in accordance with example embodiments.

FIGS. 9D and 9E depict aspects of a model development and release cycle, in accordance with example embodiments.

FIG. 10 depicts a distributed architecture for scheduling and computing genetic groupings, as well as processing and storing the associated results, in accordance with example embodiments.

FIG. 11 is a flow chart, in accordance with example embodiments.

FIG. 12 is a flow chart, in accordance with example embodiments.

FIG. 13 is a flow chart, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless stated as such. Thus, other embodiments can be utilized and other changes can be made without departing from the scope of the subject matter presented herein.

Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations. For example, the separation of features into “client” and “server” components may occur in a number of ways.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

The following U.S. patents and U.S. patent applications are incorporated by reference herein in their entirety: 10,296,847, 10,755,805, 10,854,318, 11,031,101, 2021/0020266, and 2022/0051751. However, such incorporation by reference is not an admission that the incorporated document is prior art with respect to the present disclosure.

### I. Definitions

The following terms are generally used as defined below. Nonetheless, given that many words have more than one meaning, the context in which such terms are used may modify their meanings accordingly.

The term “individual” refers to a single human person or organism for which genetic data is known or can be obtained.

The terms “group” or “genetic group” refers to one or more such individuals who are genetically linked in some fashion by their IBD segments. The clustering of individuals into groups may be probabilistic in nature.

The term “population” refers to some or all contributors to a genetic dataset stored in one or more databases. Thus, a “population” could contain a large number of individuals who have been clustered into groups, as well as ungrouped individuals.

The term “end user” refers to a person who uses a genetic computing platform to make queries and/or view results related to genetic data. This genetic data may be that of the end user, another individual, or a group.

The terms “genetic data” and “genetic datasets” refer to digitized representations of sequenced or partially sequenced genomes of individuals.

The term “genetic computing platform” refers to one or more computing devices or systems (e.g., processing, storage, and/or communication hardware and/or software) that are configured to carry out the embodiments herein.

The term “reference panel” refers to a set of individuals whose genome is typical of those from particular region, e.g., people native to a place or belonging to a genetic group.

The term “haplotype” refers to a set of genomic polymorphisms that are physically located near one another on a chromosome and tend to be inherited together.

The terms “recent ancestor clustering” and its acronym “RAC” refers to a set of algorithms that assign individuals to genetic groups (which are more granular than political or administrative regions) based on IBD clustering results. Nonetheless, RAC algorithms can also map each of these genetic groups to one or more political or administrative regions in some cases.

### II. Example Computing Devices and Cloud-Based Computing Environments

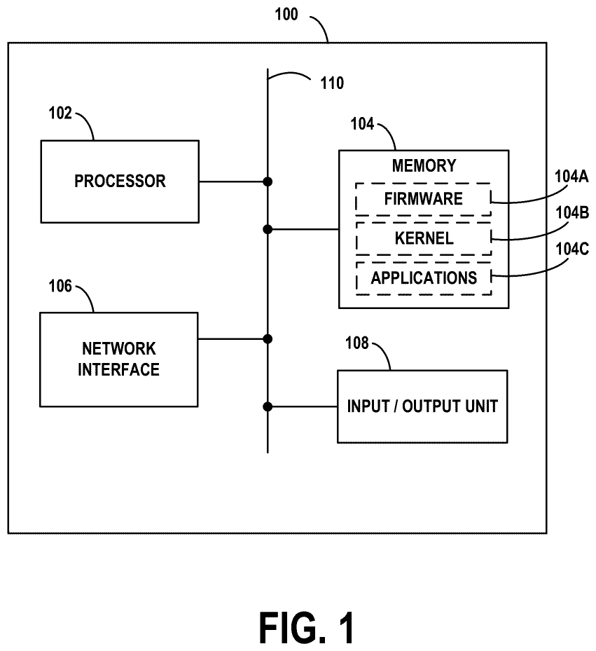

FIG. 1 is a simplified block diagram exemplifying a computing device 100, illustrating some of the components that could be included in a computing device arranged to operate in accordance with the embodiments herein. Computing device 100 could be a client device (e.g., a device actively operated by a user), a server device (e.g., a device that provides computational services to client devices), or some other type of computational platform. Some server devices may operate as client devices from time to time in order to perform particular operations, and some client devices may incorporate server features.

In this example, computing device 100 includes processor 102, memory 104, network interface 106, and input/output unit 108, all of which may be coupled by system bus 110 or a similar mechanism. In some embodiments, computing device 100 may include other components and/or peripheral devices (e.g., detachable storage, printers, and so on).

Processor 102 may be one or more of any type of computer processing element, such as a central processing unit (CPU), a co-processor (e.g., a mathematics, graphics, or encryption co-processor), a digital signal processor (DSP), a network processor, and/or a form of integrated circuit or controller that performs processor operations. In some cases, processor 102 may be one or more single-core processors. In other cases, processor 102 may be one or more multi-core processors with multiple independent processing units. Processor 102 may also include register memory for temporarily storing instructions being executed and related data, as well as cache memory for temporarily storing recently-used instructions and data.

Memory 104 may be any form of computer-usable memory, including but not limited to random access memory (RAM), read-only memory (ROM), and non-volatile memory (e.g., flash memory, hard disk drives, solid state drives, compact discs (CDs), digital video discs (DVDs), and/or tape storage). Thus, memory 104 represents both main memory units, as well as long-term storage. Other types of memory may include biological memory.

Memory 104 may store program instructions and/or data on which program instructions may operate. By way of example, memory 104 may store these program instructions on a non-transitory, computer-readable medium, such that the instructions are executable by processor 102 to carry out any of the methods, processes, or operations disclosed in this specification or the accompanying drawings.

As shown in FIG. 1, memory 104 may include firmware 104A, kernel 104B, and/or applications 104C. Firmware 104A may be program code used to boot or otherwise initiate some or all of computing device 100. Kernel 104B may be an operating system, including modules for memory management, scheduling and management of processes, input/output, and communication. Kernel 104B may also include device drivers that allow the operating system to communicate with the hardware modules (e.g., memory units, networking interfaces, ports, and buses) of computing device 100. Applications 104C may be one or more user-space software programs, such as web browsers or email clients, as well as any software libraries used by these programs. Memory 104 may also store data used by these and other programs and applications.

Network interface 106 may take the form of one or more wireline interfaces, such as Ethernet (e.g., Fast Ethernet, Gigabit Ethernet, and so on). Network interface 106 may also support communication over one or more non-Ethernet media, such as coaxial cables or power lines, or over wide-area media, such as Synchronous Optical Networking (SONET) or digital subscriber line (DSL) technologies. Network interface 106 may additionally take the form of one or more wireless interfaces, such as IEEE 802.11 (Wifi), BLUETOOTH®, global positioning system (GPS), or a wide-area wireless interface. However, other forms of physical layer interfaces and other types of standard or proprietary communication protocols may be used over network interface 106. Furthermore, network interface 106 may comprise multiple physical interfaces. For instance, some embodiments of computing device 100 may include Ethernet, BLUETOOTH®, and Wifi interfaces.

Input/output unit 108 may facilitate user and peripheral device interaction with computing device 100. Input/output unit 108 may include one or more types of input devices, such as a keyboard, a mouse, a touch screen, and so on. Similarly, input/output unit 108 may include one or more types of output devices, such as a screen, monitor, printer, and/or one or more light emitting diodes (LEDs). Additionally or alternatively, computing device 100 may communicate with other devices using a universal serial bus (USB) or high-definition multimedia interface (HDMI) port interface, for example.

One or more computing devices like computing device 100 may be deployed to support the embodiments herein. The exact physical location, connectivity, and configuration of these computing devices may be unknown and/or unimportant to client devices. Accordingly, the computing devices may be referred to as “cloud-based” devices that may be housed at various remote data center locations.

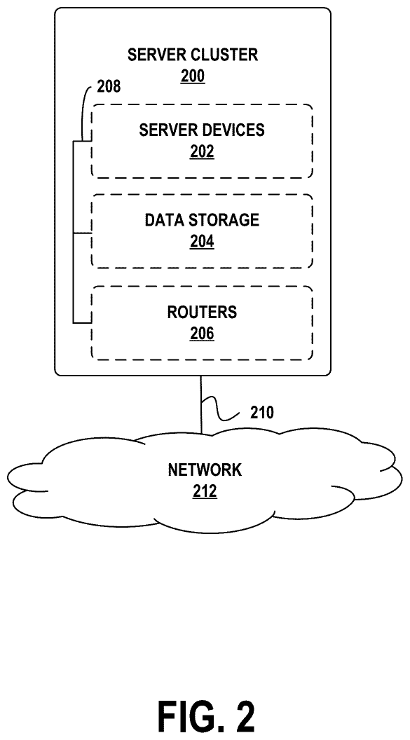

FIG. 2 depicts a cloud-based server cluster 200 in accordance with example embodiments. In FIG. 2, operations of a computing device (e.g., computing device 100) may be distributed between server devices 202, data storage 204, and routers 206, all of which may be connected by local cluster network 208. The number of server devices 202, data storages 204, and routers 206 in server cluster 200 may depend on the computing task(s) and/or applications assigned to server cluster 200.

For example, server devices 202 can be configured to perform various computing tasks of computing device 100. Thus, computing tasks can be distributed among one or more of server devices 202. To the extent that these computing tasks can be performed in parallel, such a distribution of tasks may reduce the total time to complete these tasks and return a result. For purposes of simplicity, both server cluster 200 and individual server devices 202 may be referred to as a “server device.” This nomenclature should be understood to imply that one or more distinct server devices, data storage devices, and cluster routers may be involved in server device operations.

Data storage 204 may be data storage arrays that include drive array controllers configured to manage read and write access to groups of hard disk drives and/or solid state drives. The drive array controllers, alone or in conjunction with server devices 202, may also be configured to manage backup or redundant copies of the data stored in data storage 204 to protect against drive failures or other types of failures that prevent one or more of server devices 202 from accessing units of data storage 204. Other types of memory aside from drives may be used.

Routers 206 may include networking equipment configured to provide internal and external communications for server cluster 200. For example, routers 206 may include one or more packet-switching and/or routing devices (including switches and/or gateways) configured to provide (i) network communications between server devices 202 and data storage 204 via local cluster network 208, and/or (ii) network communications between server cluster 200 and other devices via communication link 210 to network 212.

Additionally, the configuration of routers 206 can be based at least in part on the data communication requirements of server devices 202 and data storage 204, the latency and throughput of the local cluster network 208, the latency, throughput, and cost of communication link 210, and/or other factors that may contribute to the cost, speed, fault-tolerance, resiliency, efficiency, and/or other design goals of the system architecture.

As a possible example, data storage 204 may include any form of database, such as a structured query language (SQL) database. Various types of data structures may store the information in such a database, including but not limited to tables, arrays, lists, trees, and tuples. Furthermore, any databases in data storage 204 may be monolithic or distributed across multiple physical devices.

Server devices 202 may be configured to transmit data to and receive data from data storage 204. This transmission and retrieval may take the form of SQL queries or other types of database queries, and the output of such queries, respectively. Additional text, images, video, and/or audio may be included as well. Furthermore, server devices 202 may organize the received data into web page or web application representations. Such a representation may take the form of a markup language, such as HTML, the eXtensible Markup Language (XML), or some other standardized or proprietary format. Moreover, server devices 202 may have the capability of executing various types of computerized scripting languages, such as but not limited to Perl, Python, PHP Hypertext Preprocessor (PHP), Active Server Pages (ASP), JAVASCRIPT®, and so on. Computer program code written in these languages may facilitate the providing of web pages to client devices, as well as client device interaction with the web pages. Alternatively or additionally, JAVA® may be used to facilitate generation of web pages and/or to provide web application functionality.

### III. Recent Ancestor Clustering

Geneticists, scientists, medical professionals, and individuals may have various reasons to want to be able to cluster individuals into genetic groups. For instance, such groupings may be helpful in understanding genetic relationships, migratory patterns, language evolution, and/or predispositions for certain diseases or health conditions. Other benefits, such as gaining further insight into one's ancestry may be desirable or beneficial.

But today's genetic computing platforms define genetic groups based more prominently on political and administrative regions (e.g., countries, states, provinces, counties, etc.) than on shared genetic ancestry. Doing so obscures potentially relevant information relating to migrations, diasporas, languages, and genetic linkages. Further, the accuracy of mappings of these genetic groups to political and administrative regions can vary dramatically. For example, some genetic groups may have occupied only a small part of a much larger political or administrative region, but are associated with the entire region.

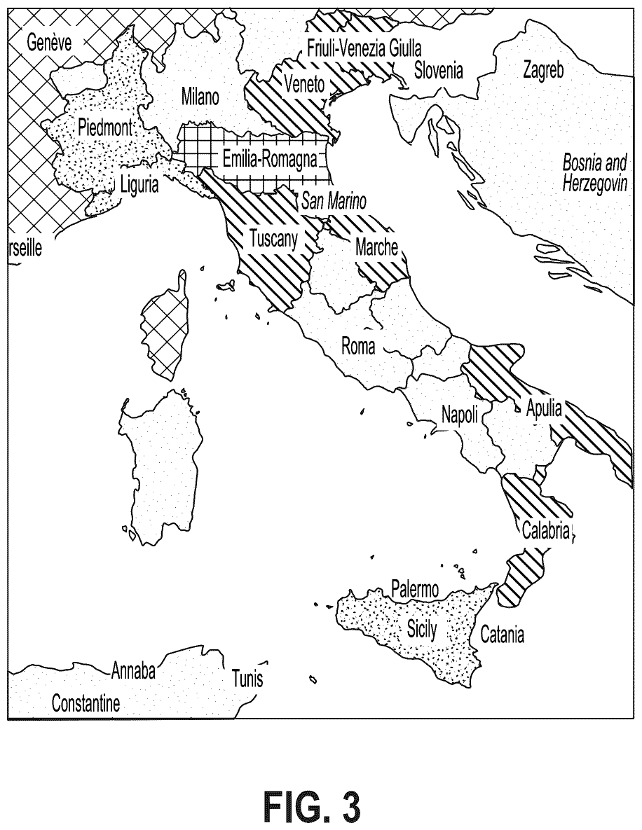

As an example of these deficiencies, FIG. 3 depicts a hypothetical clustering of an individual's genetic data within genetic groups defined by a political map of Italy. The administrative regions of Italy are shaded based on strengths of linkage between the individual's genetic data and those of the respective groups found within these regions, with darker shadings representing stronger links. Nonetheless, this clustering is coarse in nature, and does not represent how genetic groups may migrate or how political maps can be redrawn over time. For instance, Sicily may have been the ancestral home of several distinct genetic groups each located in their own sub-region, but such information is obscured by existing techniques.

In contrast, the embodiments herein can achieve fine-grained mappings of individuals to genetic groups, where those genetic groups are associated with geographic regions that more accurately reflect actual migration, language use, ethnicity, and/or other possible cultural indicators. Such geographic regions can be arbitrarily defined based on historical and genetic data, and thus are not necessarily based on political or administrative boundaries. Nonetheless, these embodiments can associate certain genetic groups with political or administrative regions.

Further, these embodiments use a custom machine learning pipeline, as well as other technical advances, in order to perform operations related to genetic grouping in a manner that reduces the magnitude of computing resources (e.g., processing, memory, long-term storage, and/or communication) thus making the genetic computing platform more scalable.

This improvement in performance is important, as updates to genetic group clustering may be carried out several times per year, as the genetic datasets continue to grow. Furthermore, the ability to rapidly generate new models and classifiers allows researchers to explore and evaluate improvements to how individuals are assigned to genetic groups.

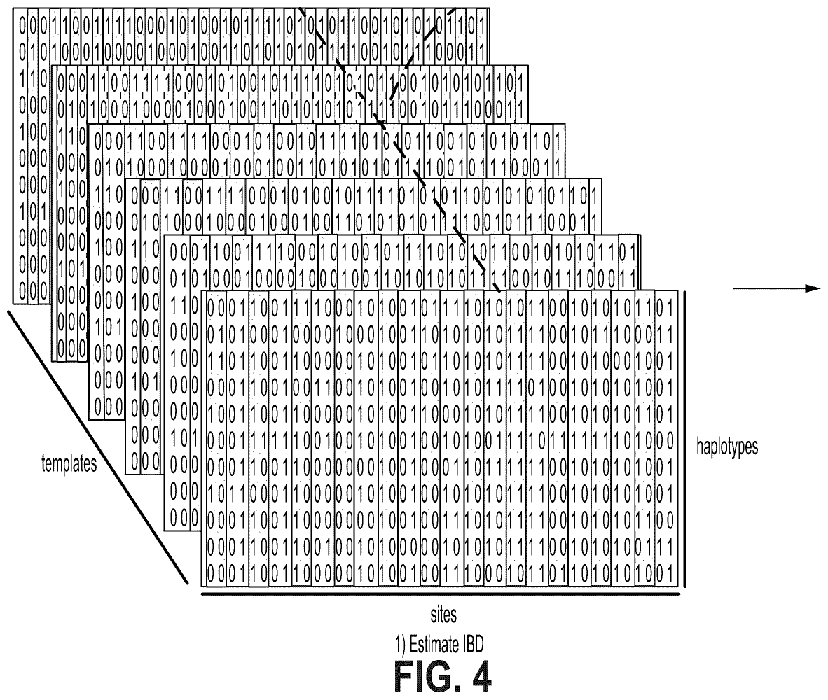

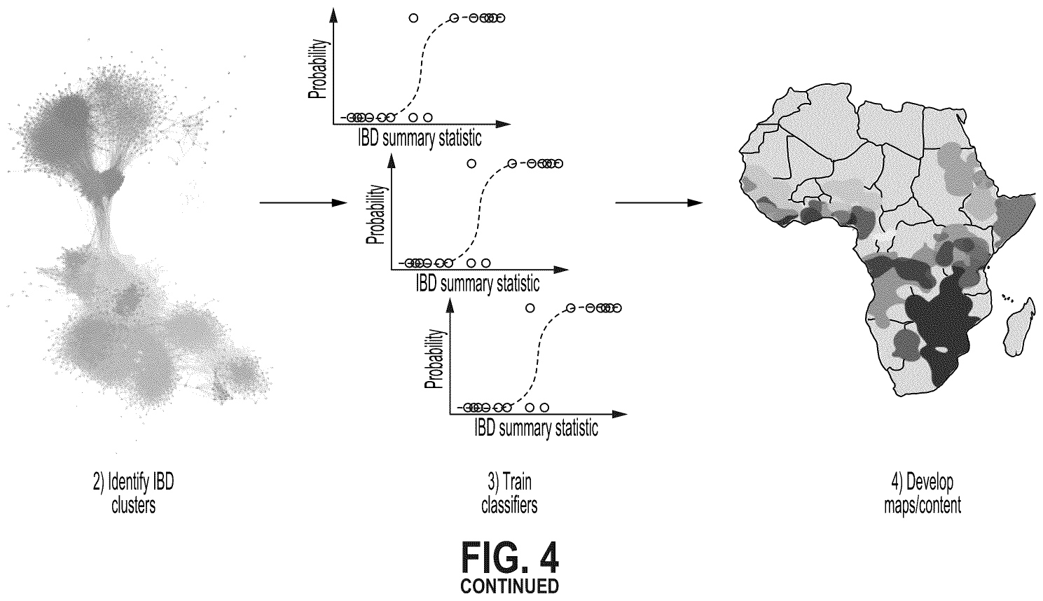

FIG. 4 provides a high-level overview of this process. Step 1 involves estimating IBD for a population of individuals for whom genetic data is available to the genetic computing platform. Step 2 involves identifying IBD clusters, preferably using SBMs. Step 3 involves training a plurality of classifiers to make predictions of which genetic groups any given individual is likely to belong. Step 4 involves developing maps of geographical regions associated with these genetic groups, as well as content (e.g., text, images, audio, video) describing the groups. Each of these steps is described in more detail below. Importantly, implementations of the embodiments herein may not draw major distinctions between certain steps. For instance, steps 1, 2, and 3 may be parts of an automated machine learning pipeline, while step 4 may combine computing and manual input from human experts.

Unless indicated otherwise, a genetic computing platform may include one or more computing devices, such as computing device 100, and/or one or more server clusters, such as server cluster 200. Other arrangements are possible.

A. Estimating IBD

As discussed above, IBD refers to matching genomic segments shared by a plurality of individuals that were inherited from a common ancestor. IBD segments are considered to be matches when the alleles on a paternal or maternal chromosome exhibit similarity above some pre-determined threshold. The length of IBD segments can vary based on the number of generations between the individuals and the ancestor (e.g., longer segments tend to be found when the common ancestor is recent than when the common ancestor is not recent).

Estimating IBD segments is challenging not only due to the size of genomic datasets but also due to two types of errors that break up IBD segments: genotyping and phase switch errors. Genotyping error occurs when the observed genotype of an individual is miscalled due to sequencing or microarray errors. Phase switch errors occur when alleles are assigned to the incorrect haplotype within a diploid individual during statistical phasing. Moreover, IBD segments may contain discordant alleles due to mutation or gene conversion since the common ancestor.

Together, these errors and discordances may lead IBD inference methods to fragment true long IBD segments into many shorter, erroneous segments on separate haploid chromosomes. Some of these short fragments of IBD may be below the minimum segment length at which IBD inference methods can reliably make estimates. This can then result in an underestimate of the total proportion of the genome that is IBD because short fragments may be erroneously discarded as false IBD. Additionally, the number of IBD segments shared between the two individuals may be overestimated when a fragmented long IBD segment is erroneously identified as several shorter segments.

While a number of IBD estimating techniques are available, the embodiments herein may make use of the phasedIBD approach. This algorithm achieves low false positive rates for short IBD segments, which is desirable for detecting granular group structure. Nonetheless, other IBD estimating techniques may be used.

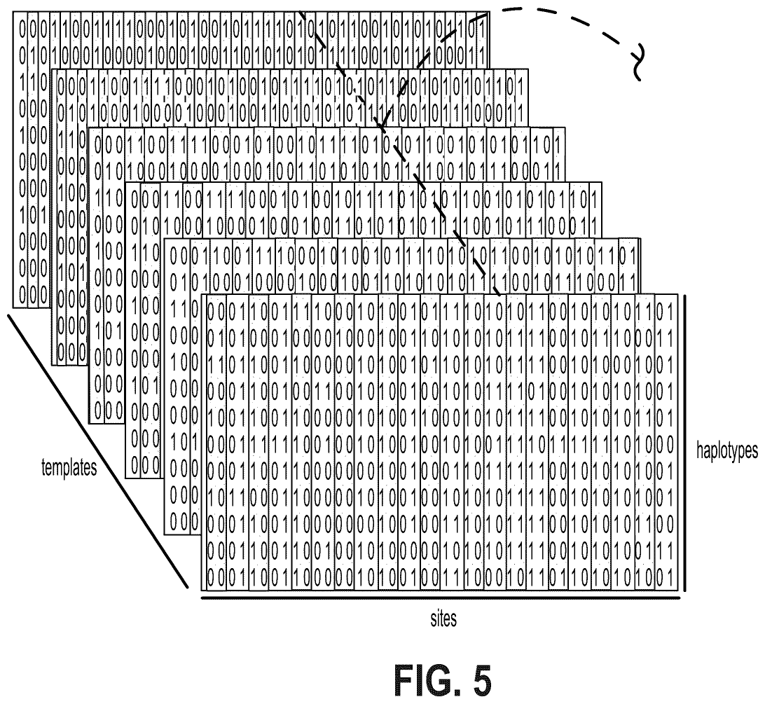

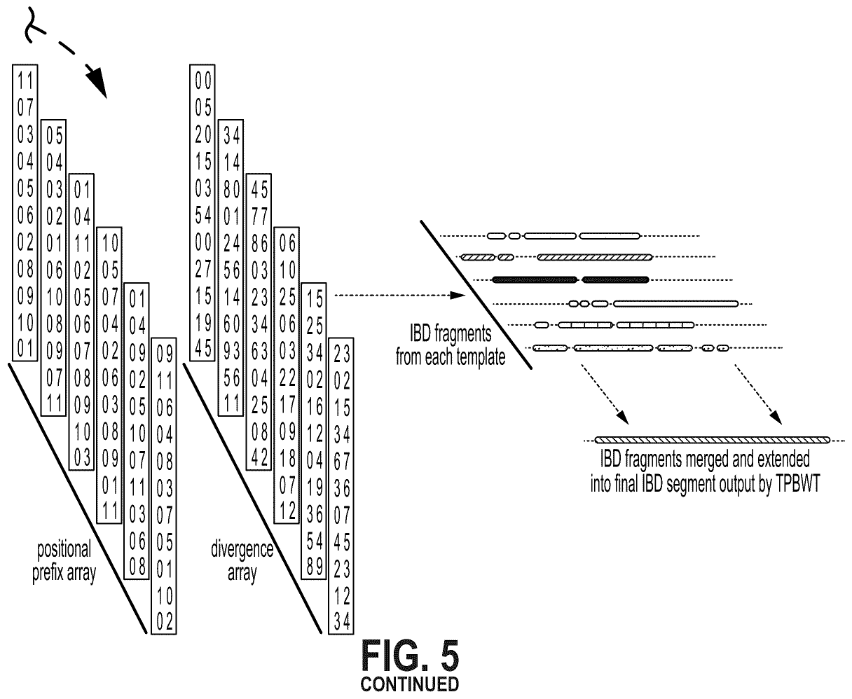

The phasedIBD algorithm involves employing the templated positional Burrows-Wheeler transform (TPBWT) to make fast IBD estimates that are robust to genotype and phasing errors. Data manipulated by the algorithm is shown in FIG. 5.

To identify haplotype sharing among a large panel of haplotypes, TPBWT passes once through an M by N by t 3D structure, where M is the number of haplotypes, N is the number of biallelic sites, and t is the number of templates. Each template is a pattern at which sites are masked out (e.g., shaded in FIG. 5).

During the left to right pass through this structure, at each site k, two arrays are updated (curved arrow). The positional prefix array (PPA) and the divergence array (DIV) are both 2D arrays of size M by t. At site k, each of the t columns of PPA and DIV are updated for the templates that are not masked out. Each of the t columns in PPA contains the haplotypes sorted in order of their reversed prefixes. Similarly, each of the t columns in DIV contains the position at which matches began between haplotypes adjacent to one another in the sorted order of PPA.

During the left to right pass through this structure, short fragments of IBD shared between haplotypes i and j, broken up by errors, are identified by each of the t templates (straight arrows). As these fragments are identified, they are merged and extended with one another in the current match arrays Pₛ and Pₑ.

While merging and extending IBD fragments, a heuristic is used to scan for and fix putative phase switch errors. When a phase switch error is identified using one template, the haplotypes of the individual are swapped in all future sites visited by all templates. Thus, phase switch errors identified in one template effectively modifies the ordering of haplotypes in the positional prefix arrays of the other templates; this dependency across templates means that TPBWT identifies and merges together fragments of identical-by-state segments that may not have been identified in the first place. Moreover, this means that phase switch errors are fixed consistently throughout the entire cohort—phase switch errors corrected in one individual are consistent across all the IBD that individual shares with all other individuals. This consistency helps ensure that IBD segments can be correctly triangulated within the cohort; if individual A shares a segment with individual B, and individual A shares an overlapping segment with individual C, then individuals B and C should also share an overlapping segment.

B. Stochastic Block Models for Inference of Genetic Group Structure

SBMs are probabilistic generative models for graphs. They are a highly flexible approach for modeling the structure of a graph. The embodiments herein may use a special case of the general SBM called the planted partition model, which identifies assortative structure within a graph. Assortative structure is present when the graph can be partitioned into groups in which vertices of the same group are more likely to be connected by an edge than vertices of different groups. As noted, use of SBM for inferring genetic group structure has not previously been done, and doing so provides significant savings of computing resources.

Once IBD segments are identified, they can be arranged to form a graph that includes the inferred genetic linkages between individuals. The next step may involve identifying genetic groups within this graph. Formally, the IBD estimates are used to construct the IBD graph A, in which each vertex represents an individual and the number of edges Aᵢⱼ between vertices i and j are calculated as:

Aᵢⱼ=min(β, nᵢⱼ)/z
In this equation nᵢⱼ is the amount of IBD in centimorgans shared between individuals i and j, z is a divisor that adjusts how the pairwise IBD is weighted in the IBD graph, and β is the maximum amount of IBD that will be considered for a pairwise relationship. It is desirable to estimate population structure and not just close family structure. Thus setting β to a value in the range of 100-500 centimorgans masks out the signal of close family relationships in the IBD graph. The divisor z can take on any value greater than 0. If z is set to 1.0, then the number of edges connecting two individuals is equal to the number of centimorgans they share in IBD segments. Larger values of z make connections between all individuals more evenly weighted regardless of how much IBD these individuals share.

Centimorgans are a unit of genetic linkage based on the distance between chromosome positions for which the average number of intervening chromosomal crossovers in a single generation is 1%. One centimorgan corresponds to roughly 1 million base pairs in the human genome, but can vary based on the location in the genome that is being considered.

It is desirable to infer assortative structure within an IBD graph in which genetic groups consist of individuals who share more IBD with one another than with individuals in other groups. The amount of IBD shared between two individuals is measured as the value Aᵢⱼ, which is calculated by the sum of the length of the IBD segments. Using Bayesian inference, the vector of assignments of individuals to genetic groups, b, can be determined given the observed IBD graph A:

P(b|A)=P(A|b)P(b)/P(A)

In any given sample taken during the MCMC procedures described below, each individual is assigned to only one group. That being said, the marginal posterior probabilities of the vector P(b|A) can indicate that individuals are in more than one group. As just one possible example, P(b|A) can be interpreted as “an individual is assigned to group 1 with 0.4 probability and group 2 with 0.6 probability,” which may mean the individual is admixed between the two groups.

For the planted partition stochastic block model, the likelihood of observing the IBD graph A given the assignment of individuals to genetic groups b is given by:

$$\\ P ( A \vert b ) = ( {e}_{in} ! {e}_{out} ! ) / [ {( \frac{B}{2} )}^{{e}_{in}} {( \frac{B}{2} )}^{{e}_{out}} ( E + 1 ) ] \times {\prod }_{r} ( \frac{( {n}_{r} - 1 ) !}{( {e}_{r} + {n}_{r} - 1 ) !} ) \times \frac{{\prod }_{i} {k}_{i} !}{{\prod }_{i < j} {A}_{ij} ! {\prod }_{i} {A}_{ii} !!}$$

In this likelihood, B is the number of genetic groups, E is the total number of edges in A, eᵢₙ is the total number of edges within genetic groups, eₒᵤₜ is the total number of edges between different genetic groups, eᵣ is the number of edges between individuals within genetic group r, nᵣ is the number of individuals in genetic group r, and kᵢ is the degree of individual i. To perform inference, the prior probability of the assignment of individuals to genetic groups can be calculated, where N is the total number of individuals:

$$\\ P ( b ) = \frac{{\prod }_{r} {n}_{r} !}{N !} {( \frac{N - 1}{B - 1} )}^{- 1} {N}^{- 1}$$

The posterior distribution of genetic group assignments is estimated by sampling group assignments using Markov chain Monte Carlo (MCMC), which avoids computing the intractable normalization term P(A). MCMC algorithms sample from a probability distribution that is represented by a Markov chain. The MCMC algorithm used herein employs merge-split proposals. For example, each training individual can be assigned to the genetic group maximizing the marginal posterior distribution of assignments for that individual. Individuals whose maximum marginal posterior assignment probability is less than some significance threshold (e.g., 0.99) may be removed. Genetic groups having fewer than some minimum number of assigned individuals may also be removed.

Once genetic groups have been identified during the IBD clustering analysis, an ensemble of logistic regression classifiers are trained to make assignments of individuals to each genetic group. For each individual i and each cluster r the following statistic is computed:

$$\\ {λ}_{ir} = \frac{{\sum }_{{n}_{r}} D}{{( {α}_{i} )}^{c} \times G}$$

Where aᵢ is the genome-wide ancestry proportion of an appropriate reference group for individual i. The value of aᵢ can be calculated using a separate Ancestry Composition algorithm, and may take on a value of 0.0 to 1.0, for example. If aᵢ is below some given threshold (e.g., 0.001 though other thresholds could be used) then λᵢᵣ=0. The term c ε[0,1] is a scaling factor that determines how strongly aᵢ is weighted in the final statistic, and G is the diploid length of the human genome in centimorgans (i.e., 7194.4). The numerator is the sum of merged IBD segments D in centimorgans for all nᵣ individuals with a MAP assignment to genetic group r. The IBD segments are merged such that any genetic regions covered by overlapping IBD segments are only counted once.

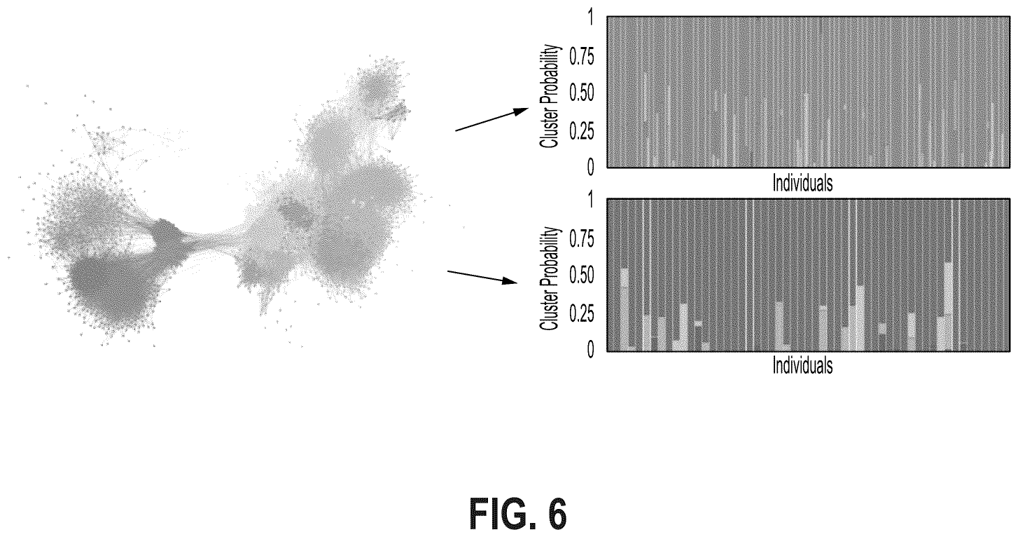

This result is represented visually in FIG. 6. Each cluster found in the graph on the left side of FIG. 6 contains individuals who have been determined to have at least a threshold probability of being in that cluster. Thus, as shown in the right side of FIG. 6, the process results in each individual having a distinct probability of being in each cluster.

C. Classifier Training

Using A ir as model features and the MAP genetic group assignments as training data observations, logistic regression classifiers are trained for each of the r genetic groups. Leave-one-out cross validation (or another cross-validation technique) can be used to evaluate model performance.

For purposes of illustration (and using generic variables), the log-odds 1 of an outcome p=P(Y=1) for an output variable Y can be modeled with a logistic function over independent variables x₁ and x₂ as:

$$\\ l = ln \frac{p}{1 - p} = {β}_{0} + {β}_{1} {x}_{1} + {β}_{2} {x}_{2}$$

Thus, the probability that Y=1 can be written as:

$$\\ p = \frac{1}{1 + {e}^{- ( {β}_{0} + {β}_{1} {x}_{1} + {β}_{2} {x}_{2} )}}$$

Once the values for βᵢ are found, the probabilities that Y=1 and Y=0 is easily derived. The values for βᵢ can be found from performing a maximum likelihood estimation on the training data. This may entail, for example, using an iterative process, such as Newton's method, until the values converge. Various techniques can be used to determine a threshold above which a predicted probability indicates that an individual belongs to a genetic group. Such thresholds may vary per group.

Notably, logistic regression with other than two independent variables may be used. Also, a classifier technique other than logistic regression can be used. For example, classifiers based on linear regression, polynomial regression, decision trees, or neural networks are possible.

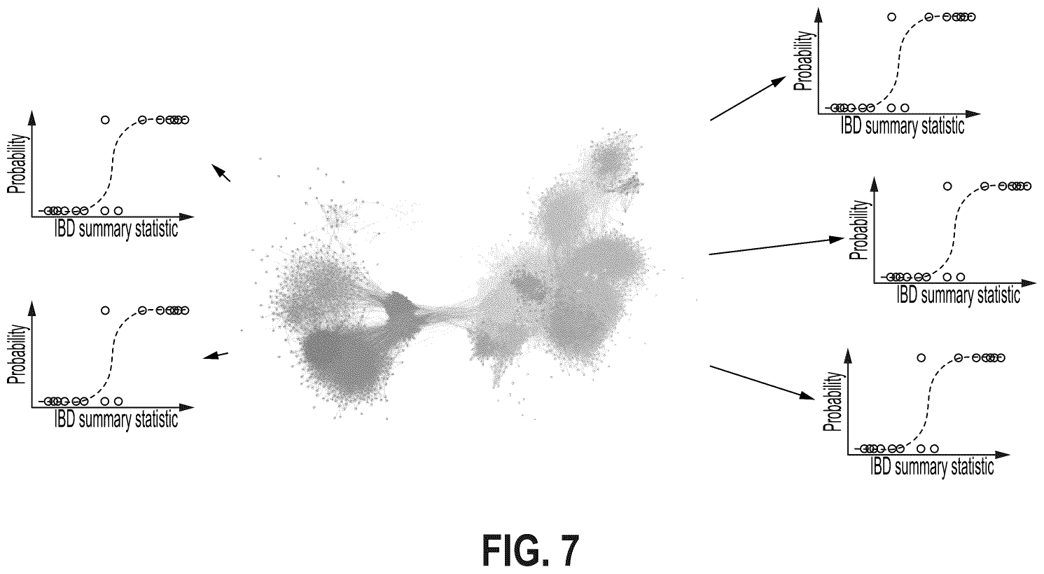

This process is illustrated in FIG. 7. Each cluster in the graph represents a distinct genetic group, data from which is used to train a classifier for that group. When presented with IBD data of an individual (e.g., a new individual whose genetic data was not used in the training process), such a classifier may return a probability that the individual belongs to the group. This probability may relate to a confidence that the individual belongs to the group, where a higher probability indicates more confidence. Alternatively, the classifier may return a binary value indicating whether or not the individual is predicted to belong to the group.

D. Developing Geographic Maps and Content

Maps and further content can be developed based on these highly-granular associations between individuals and genetic groups. Advantageously, the maps can be based on arbitrary geographic locations and/or boundaries rather than political or administrative regions. Kriging and/or kernel density estimate algorithms can be used to generate map shapes. Both map content and user-submitted content/feedback can be used to develop labels. For instance, subject matter experts, geneticists, and historians may be consulted to develop ethnolinguistic labels for each genetic group, and these labels may be refined based on end user feedback.

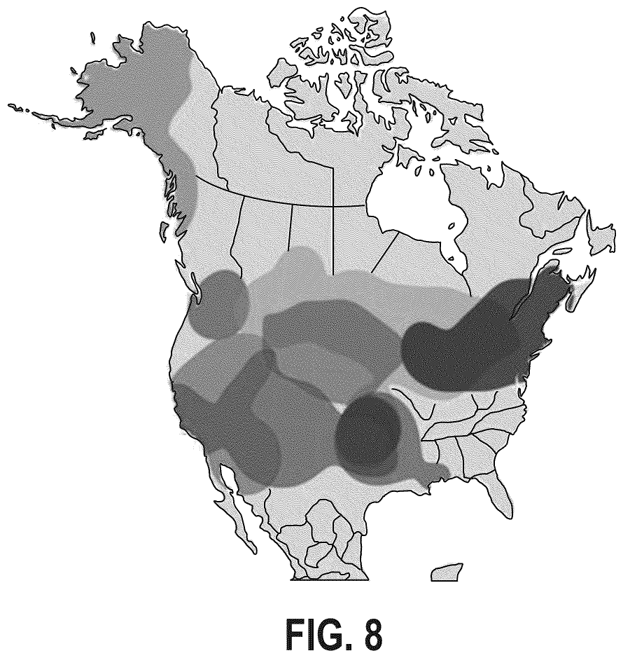

An example of such an improved map is shown in FIG. 8. This map depicts overlapping geographic regions for certain genetic groups associated with Indigenous Americans. Unlike earlier maps, this map does not rely on country, state, or provincial boundaries.

### IV. Architecture and Pipelines

Various architectures and pipelines may be used in order to enable, facilitate, support, and/or implement the embodiments herein. These architectures and pipelines may take the form of a plurality of software modules that are executed in a particular order (with some degree of parallelism possible) by one or more computing devices of a genetic computing platform, as well as data used by these software modules.

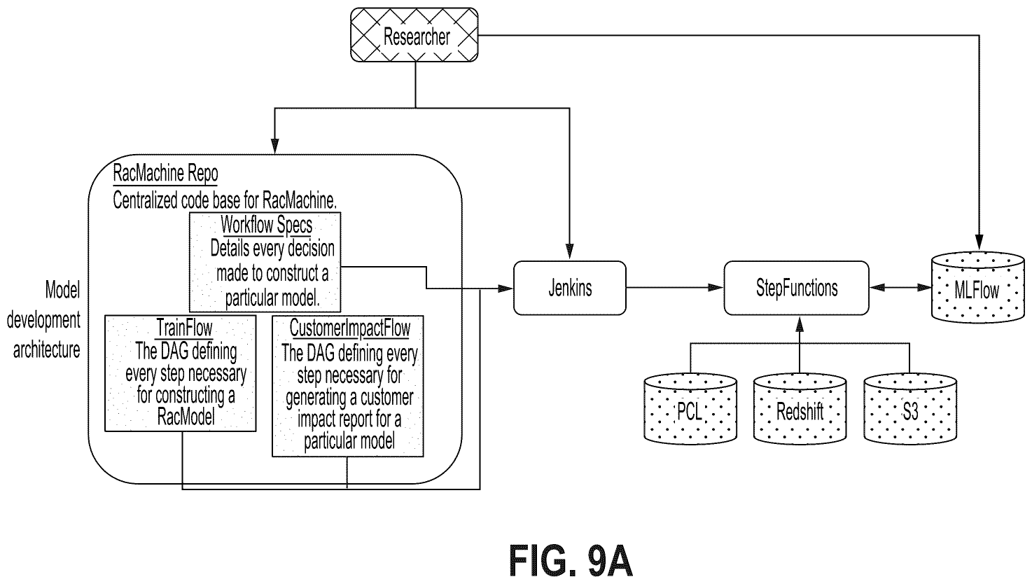

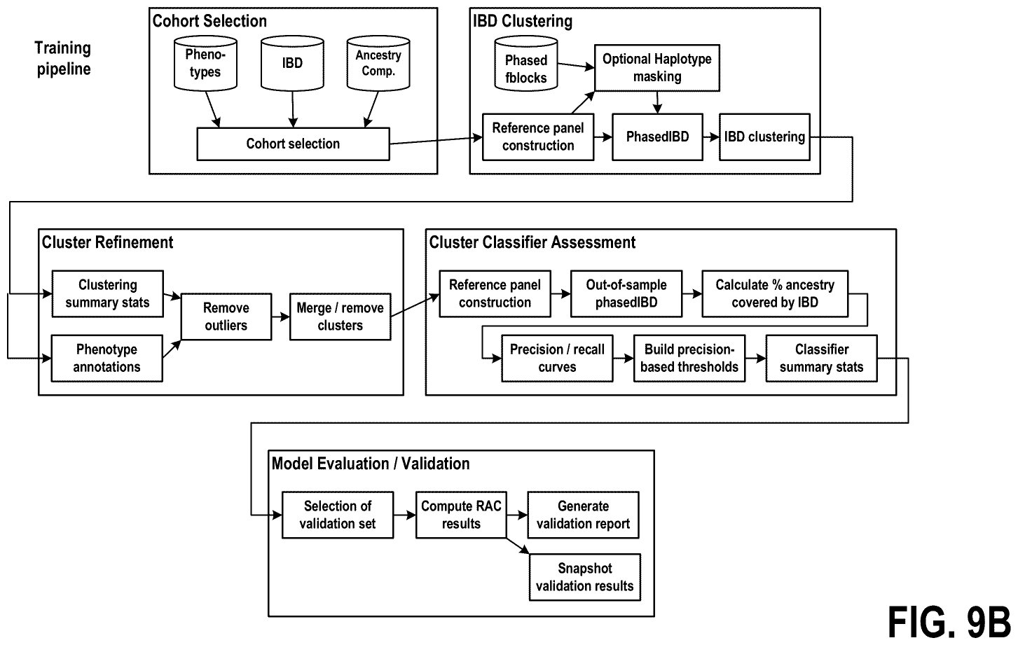

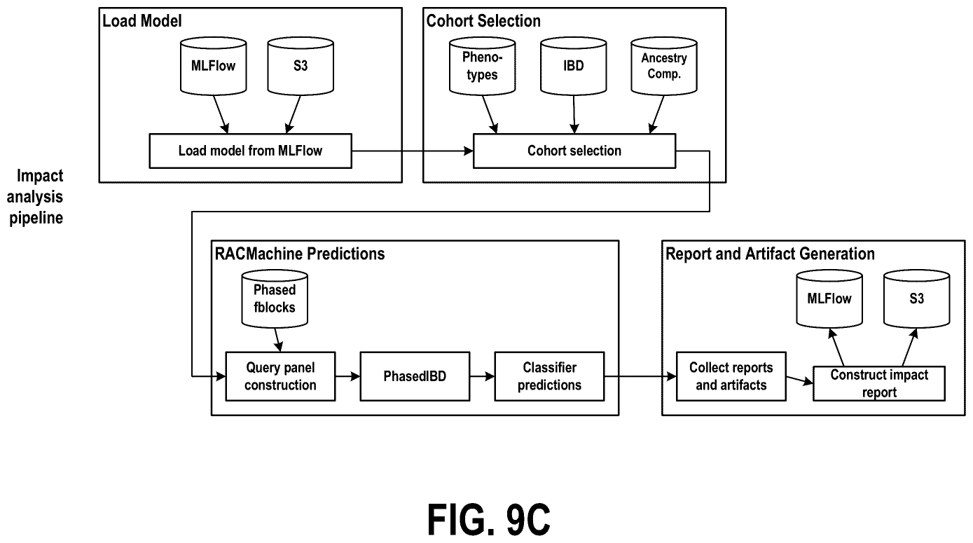

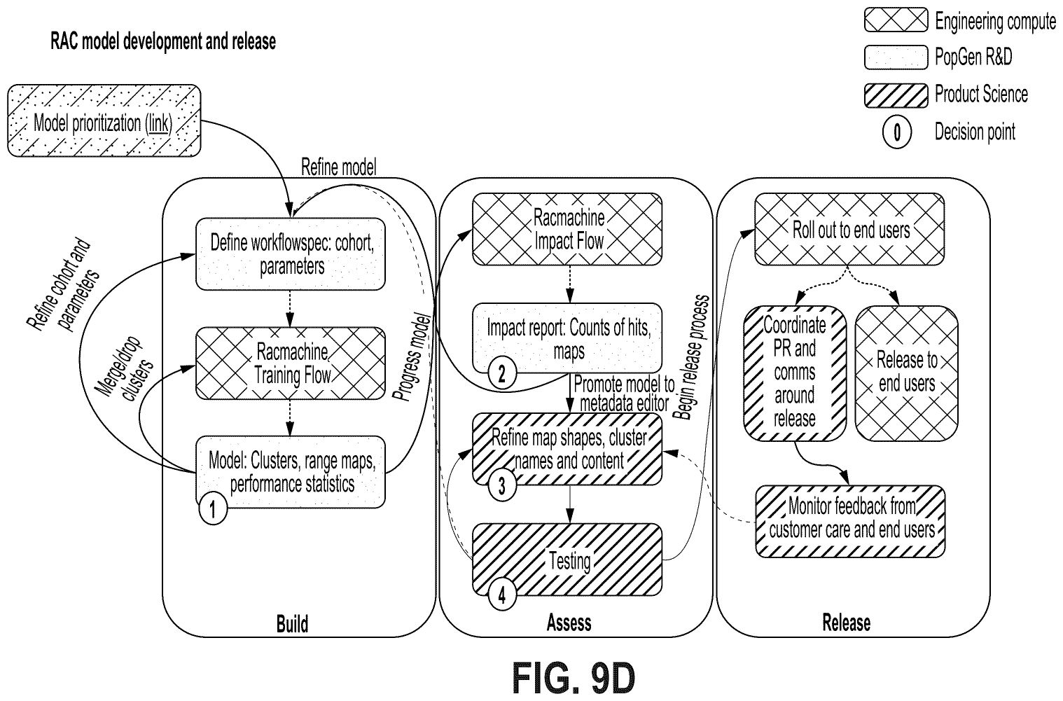

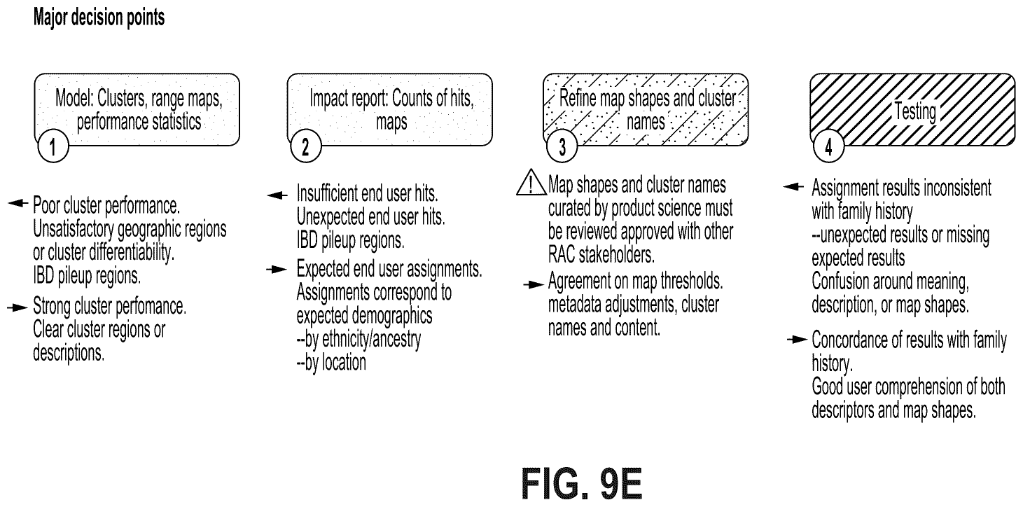

FIG. 9A provides overview of an architecture for model development, FIG. 9B provides a pipeline for training, and FIG. 9C provides a pipeline for impact analysis. Nonetheless, other pipelines, including variations of these pipelines may be used with the embodiments herein.

A. Model Development Architecture

The model development architecture may include the following components.

RacMachine Repository: The code for the entire project, including workflow definitions, datastore access, and computation logic, can be contained within a singular repository to which researchers have access. Researchers can create feature branches in this repository where they can define their unique experiments through a workflow specification. Afterward, they can schedule their training runs using Jenkins.

Jenkins: Jenkins can serve as the common interface for researchers to specify their feature branch, their workflow specification, and whichever flow they want to initiate.

Workflow Specification: This can be defined in YAML, XML, JSON, or a Python configuration file that encapsulates all decisions that need to be made during the course of a training run. This includes a language for defining how a cohort is constructed in addition to how the subsequent clustering algorithm is run.

MLFlow: A service used for tracking and storing artifacts for each completed training run. These artifacts include reports that are generated automatically in addition to the trained model that can potentially be used to serve predictions.

Metaflow: An orchestration tool used to manage running workflows in Amazon Web Services (AWS). This tool interfaces directly with AWS resources like StepFunctions, and Batch so that multiple researchers can run multiple distributed training runs in parallel. On top of this tool, additional mechanisms support workflow resumability so that researchers can make iterative improvements without needing to rerun their flows from scratch every time.

AWS Batch: An AWS service that handles running large scale distributed computes.

AWS Redshift: A cloud data warehouse.

AWS S3: An object storage service that will hold the models/artifacts necessary for RAC computations.

AWS StepFunctions—A workflow scheduling system that handles traversing the training DAG.

B. Training Pipeline

The training pipeline may include the following steps, modules, and/or data.

1. Cohort selection

Phenotypes: For more granular ancestry results, customer data can be compared to reference genotypes. Typically, individuals are used as reference genomes or as references for Ancestry Composition estimates if the customer has 2-4 grandparents from a desired region (e.g. Italy, UK, etc.). For instance, users can self-report through web interfaces that they have 4 grandparents from a specific region or as part of a specific group (e.g. Ashkenazi Jewish, etc.).

IBD: A data store that maintains IBD linkages between all individuals in the population.

Ancestry Composition: The percentage results for biogeographic ancestry estimates.

Additionally, individuals can be filtered for those that have 4 grandparents from a desired region or group.

2. IBD Clustering

Eagle is open source phasing software that is used to phase individual genomes to estimate the maternal and paternal haplotypes for the individual.

The phasedIBD algorithm includes haplotype masking based on the local Ancestry Composition results for a particular individual. For example, if a goal is to identify Ashkenazi Jewish population structure and someone is half Ashkenazi Jewish and half British, then their British haplotypes can be masked out before computing IBD. This algorithm is used to determine IBD between the individual and all individuals in the group clustered to a specific regional ancestry.

IBD overlap can be determined for the proposed cohorts for the ancestry results. Thus, IBD overlap can be found for different regional ancestries, for example different American regions shown in FIG. 8.

3. Cluster Refinement

IBD clustering: Summary statistics and phenotype annotations (e.g. tying together clusters of individuals with specific regions).

Filtering outliers: End users may have reported incorrect information or have genetics that differ from the rest of the cohort.

Merge or remove clusters: If some of the clusters have insufficient individuals or the genetics are too similar, then these clusters can be removed from the system.

4. Cluster Classifier Assessment

Reference Panel Construction.

Out-of-sample phasedIBD: this would include running multiple individuals' genotypes through the reference panels to determine the number of IBD hits there are between the individual and reference genomes, as well as the amount of IBD overlap between the individual and the reference panel. There can be IBD cutoffs for each region that are used for thresholds for the estimated strength of association between an individual and a genetic group.

Calculate percent of ancestry covered by IBD.

Precision—recall curves: This functionality involves determining the IBD thresholds that yield the most desirable precision—recall curves for a specific region.

Build precision-based thresholds: This would be the IBD hits and IBD length cut off for the results to be presented to an end user.

Classifier summary stats.

5. Model Evaluation/Validation

Selection of validation set.

Compute RAC results.

Generate validation report.

Snapshot validation results.

6. Modular Map/Report Generation (Not Shown)

Modular content preparation includes geographic maps and/or other content for each genetic group. For example, the map-based content can be automatically generated based on the cohort of individuals corresponding to four grandparents in a specific county, area, region, state, or city of a map or other specified criteria. For example, other specified criteria can be used such as grandparent birth location, end user birth location, end user current location, etc. The map-based content can also be based on kernel density estimates and optionally pre-determined kernel exclusion thresholds and/or down-weighting larger cities and overlapping kernels based on one or more population metrics. The text descriptions can be fed to the system with the system populating the modular content on the end-user facing product content.

C. Impact Analysis Pipeline

After the training workflow is run, models are stored in MLFlow. The impact these models would have on the population database if they were to be released can be evaluated using the Metaflow-based Impact Analysis Pipeline. This pipeline use another workflow specification to define a cohort of individuals. Typically, this is a random subsample of the population database, or if the goal is to study the impact on a specific subpopulation of individuals (as defined by various phenotypes, for example 2-4 grandparents from a given region) then that specific subpopulation can be randomly subsampled. IBD for cohort individuals is computed against the model's reference panel, and the resulting IBD can be used along with the individual's Ancestry Composition proportions to generate the input to the model's classifiers. Predictions are then made for each cohort individual. A wide array of reports are generated summarizing the rate of positive predictions and the confidence levels of the predictions. These statistics are produced for subcohorts of individuals stratified by their Ancestry Composition proportions and other phenotypes. Maps of the individuals' reported locations are made, and plots summarizing various phenotypic information about the individuals are generated (for example, the languages spoken by the individuals).

D. Secure Storage of Reference Panels for RAC Models

Models can be stored from a training workflow run in MLFlow. One of the components of a RAC model is the reference panel used to compute IBD. It can be constructed directly from the training cohort and it is necessary to compute IBD for new individuals against this panel in order to generate RAC results. The reference panel contains multiple haplotype alignments obtained from individual level data, such as phased sequence segments or blocks (fblocks). It is desirable to store this personal genetic data securely.

In some embodiments, role-based access is used in MLFlow to securely store the RAC model reference panels. In some embodiments, other solutions than or in addition to role-based access can be used. In one example, the reference panels for the RAC model can be stored in an AWS S3 bucket with a more restrictive access policy. For example, Metaflow provides a convenient S3 interface to store artifacts to an arbitrary S3 bucket specified when running a workflow. This can be leveraged to store the reference panel object in MLFlow's S3 bucket and store it in a separate S3 location with a more restrictive access policy. The object URL can still be stored in MLFlow as an artifact. This can be a list of S3 URLs. For example:

[
“s3://<bucket_name>/data/<flow_name>/<run_id>/panel_0.tar.gz”,
“s3://<bucket_name>/data/<flow_name>/<run_id>/panel_1.tar.gz”,
“s3://<bucket_name>/data/<flow_name>/<run_id>/panel_2.tar.gz”
]

The reference panel can be saved in smaller chunks so the reference panel object stored in S3 will be a list of smaller reference panel (XpHaplotypePanel) objects. This approach can be applied for storing other artifacts with Personal Identifiable Information (PII)/Personal Data (PD) in MLFlow in the future.

E. Model Development and Release

Aspects of the model development and release cycle are shown in FIGS. 9D and 9E.

### V. Prediction Architecture

A prediction service may be an interface between the end-user product (e.g., a web site and its associated computing infrastructure that provides end users with information related to their genetic data) and RAC results. This service can be responsible for both computing and serving results for individuals. The prediction service was designed with consideration of a number of properties. These properties includes but are not limited to: (i) phasedIBD being a core component of predicting RAC results, (ii) compute time for predictions taking on the order of minutes depending on the size of the reference panel and batch size, and (iii) resource requirements (memory/disk space) for each prediction depending on the size of the reference panel and potentially varying greatly between models.

Because of the dynamic resource requirements for predictions, results may be precomputed to ensure that end users are able to retrieve them quickly. This means that the prediction service can support: schedulable prediction (using AWS Batch), results storage (using AWS RDS), and/or a representational state transfer (REST) interface (Ploy/FastAPI) to facilitate result generation and/or retrieval.

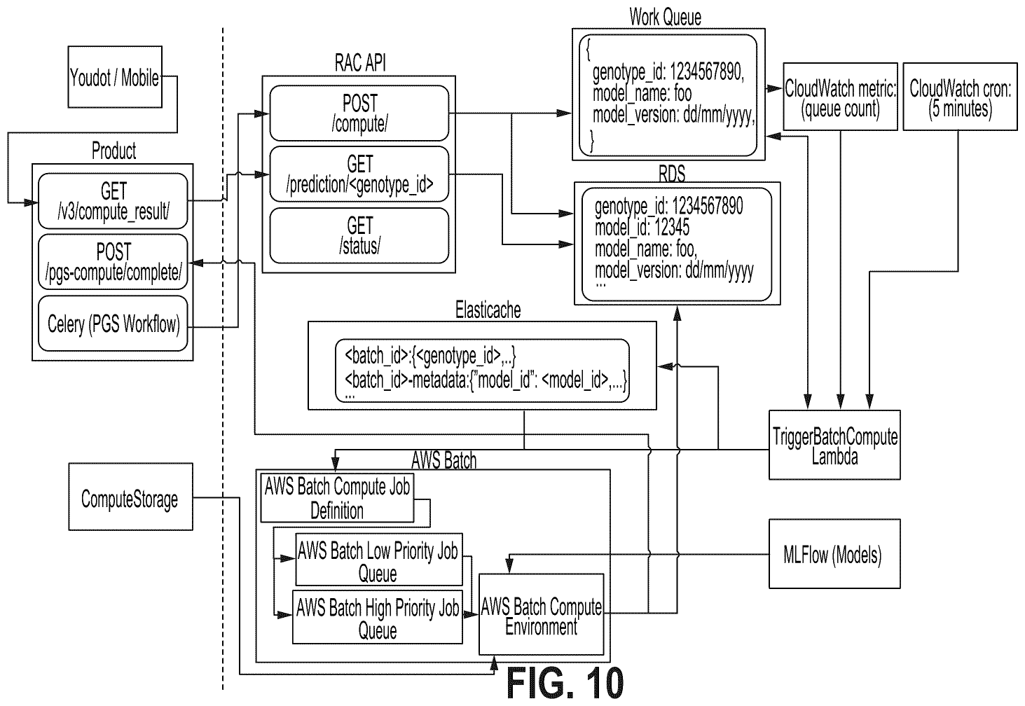

FIG. 10 depicts a computing architecture that can support such predictions. Example components of this architecture may include:

RAC API: A REST endpoint that allows other services to schedule or retrieve RAC compute results.

AWS Batch: The hardware and/or software responsible for handling the resource intensive predictions for RAC. This may be backed by an AWS Auto Scaling Group.

AWS Batch Job Definition: A job definition that specifies a particular Compute Docker Image associated with a prediction job (or another type of job) in addition to compute resource requirements.

AWS Batch Job Queue: A job queue that holds scheduled batch jobs while waiting for compute resources. Can be mapped to multiple Batch Compute Environments.

AWS Elasticache: An ephemeral datastore used to hold non-persistent orchestration data.

AWS Lambda: A simple event-based trigger that detects messages on the SQS Queue and converts them into a batch job.

AWS RDS: A relational database storage system that can hold RAC results (e.g., predictions).

AWS S3: An object storage service that can hold the models/artifacts necessary for RAC predictions or other jobs.

AWS SQS Queue: An intermediate datastore that handles buffering compute requests from the API Service.

Compute Docker Image: The docker container that contains the software responsible for handling the resource intensive computes for RAC.

MLFlow: A service used to track models (name/version) and the experiments that generated them.

RAC Model: A complete RAC model consists of a classifier, a reference panel, and additional labeling metadata for individuals in the reference panel.

A. RAC API

This service can act as the single point of interface employed by the end-user product for all RAC-related information. The API provides a consistent format when returning results and can support several endpoints.

GET/prediction/<genotype id>/− This endpoint can be used by API hosts to retrieve individual level RAC results. This endpoint can also handle rounding and filtering as necessary so that different client devices (e.g., desktop or mobile, web page or app) will retrieve consistent values. By default, this endpoint will filter responses based on model status as defined in MLFlow so that only results associated with “staged” or “promoted” models can and will be surfaced. Each individual will have at most one active entry per model name/model version.

| Query Parameters | Response |
|---|---|
| GET/prediction/<genotype_id>/ | Returns all model results for an |
| | individual. |
| | { |
| | “results”: [ |
| | { |
| | “model_id”: “foo”, |
| | “model_name”: “bar”, |
| | “model_version”: “1”, |
| | “predictions”: {...} |
| | }, |
| | ... |
| | ] |
| | } |
| GET/prediction/<genotype_id>/ | Returns specific model result(s) |
| ?model_id=[<foo>] | for an individual based on |
| | model_id(s). |
| GET/prediction/<genotype_id>/ | Returns a single model result that |
| ?model_name= | correspond to a model_name/ |
| <foo>&model_version=“bar” | model_version. |

POST/compute/—This endpoint can be used to schedule RAC prediction computes for an end user. It can asynchronously identify what computes an individual is eligible for and schedules them as part of the prediction pipeline.

| JSON Payload | Action |
|---|---|
| { | |
| “genotype_id”: 123456 | Schedules compute for all available models |
| } | for a single individual. |
| { | |
| “genotype_id”: 123456, | Schedules compute for a single individual on |
| “model_id”: [“foo”] | the one or more models. |
| } | |

GET/metadata/{model name}/{model version}. This is an endpoint that retrieves metadata associated with a specific model name and model version.

POST/prediction/. This may be a developer-only endpoint available only in non-production environments that will allow developers to inject fixtures directly into RACmachine for testing purposes.

GET/gdpr/{genotype id}. For the purpose of GDPR compliance, this endpoint will return all results for an individual.

DELETE/gdpri{genotype_id}. This endpoint triggers a GDPR deletion for an individual. For example, this process could delete all results associated with the genotype_id.

B. RAC RDS

This database can hold prediction and/or other compute job results for RAC.

| Column | Description |
|---|---|
| genotype_id | ID used to identify an individual. |
| inputs | A dictionary of input values that serve |
| | as a small snapshot of state used to |
| | generate results. |
| input_hash | A hash of the “inputs” column. |
| output | The raw compute result. |
| model_name | A human readable label for a category |
| | of RAC clusters. |
| model_version | A instance of a trained RAC. |
| active | A flag to identify an end-user's latest |
| | result for a given model_id. |

C. AWS Elasticache

This database can hold transient orchestration data for RAC predictions or other compute jobs to be stored and retrieved.

| Key | Value | Purpose |
|---|---|---|
| <batch_id> | [<genotype_id_1>, . . .] | Maps a batch ID to a list of |
| | | individuals within a particular |
| | | batch. |
| <batch_id>- | {<model_id_1>, . . .} | Maps a batch ID to relevant |
| models | | models, specifically model_id. |

D. AWS Batch

Because each RAC model can have different resource requirements, the compute environment can support scheduling different resources (virtual machines, memory, etc.). Additionally, with each new model, the compute environment can scale up to support backfills across end users.

A batching Lambda/ECS task can execute periodically and pull items off the queue. These executions may be based off a crontab scheduler, and may batch items and write placeholder records to RDS instance. Such placeholders track the inputs and batch_id of computes in progress. Batch jobs can be scheduled by batch_id.

The AWS Batch Compute Environment handles computing results for end users, can write compute results to the RAC Result database, and can signal the end-user product when an individual's predictions or other computer jobs are finished.

E. General Aspects

As noted, various aspects of a genetic computing platform may be used to facilitate and streamline the development, training, and deployment of models. Example embodiments follow, but the embodiments herein may employ different implementations.

Training pipeline: A Metaflow pipeline can be used to automate the following steps: flexibly define training cohorts, perform massive parallelized IBD computations within the cohort, perform IBD clustering analyses and post processing of genetic groups, train ensembles of logistic regression classifiers, perform cross validation, generate map shapes for each genetic groups using kernel density estimation, and produce detailed reports on the performance of the entire pipeline.

Impact pipeline: A Metaflow pipeline can be used to automate analyzing the impact of a model on existing group assignments in terms of which individuals will get new results and the quality and quantity of the new results. This is a step typically carried before choosing to promote a model for release to end users.

Model promotion: An MLFlow interface enables review of trained models and their accompanying performance reports. After reviewing the performance of a model, the model can be selected for promotion (release to end users) or may remain unavailable to end users.

Metadata GUI: After a model has been promoted, automatically generated content such as the map shapes generated by the training pipeline can be used to populate a graphical user interface. The interface enables efficiently curation of the map shapes, names, descriptions, and other content for each group within the model.

Platform API: After a model has been promoted, the genetic computing platform can request pipeline and/or model results, as well as accompanying model metadata, through a custom API. The platform API ensures updates to the presentation and content of a model made in the metadata GUI are immediately reflected in information and displays available to end users.

Model monitoring: Models can be automatically retrained, periodically or from time to time, to ensure GDPR compliance and evaluate model drift as the training dataset changes due to GDPR deletions.

### VI. Privacy Features

The genetic computing platform, in its associated architectures and pipelines, may support privacy features that allow an individual's genetic data to be deleted and/or no longer used by the predictive models. In other words, the individual's deleted genetic data might no longer be used for purposes of IBD determination, clustering, and/or training of classifiers.

These features facilitate complying with the wishes of some end users who submit their genetic data to the genetic computing platform or a related system. These end users may initially agree that their genetic data can be used for modeling and/or other purposes, but may later change their minds. Thus, it may be necessary to remove their genetic data from the platform and potentially stop using models that were based in part on their genetic data.

Furthermore, certain laws and regulations may make doing so a necessity. For example, the European Union's General Data Protection Regulation (GDPR) and the California Consumer Privacy Act of 2018 (CCPA) both provide individuals with strong protection regarding collection, user, and sharing of personal data.

The haplotype reference panel used to compute IBD is a core component of RAC and is used to provide predictions. These reference panels are constructed using individual level data, such as phased sequence segments or blocks (fblocks) directly from individuals in the population database. Different options for handling privacy issues are discussed below. The options/embodiments discussed below can be used alone or in combination with each other.

A. Surgical Removal from Reference Panels

Individual level data can be surgically removed from the reference panels in response to a deletion request. Mappings of haplotype-to-individual and individual-to-cluster are maintained and can be queried in response to a deletion request. This option is the least computationally expensive.

B. Periodic Retraining of Models

Potentially the most thorough option to ensure privacy compliance. This option additionally ensures that all models are periodically refreshed, and likely has higher computational costs than just surgical removal. Automation can be used to scale out the system to handle the periodic retraining. Training and releasing all models to end users can be done within a 30-90 day window (or other applicable window).

C. Scoped Retraining of Only Models Relating to Removed Individuals

This approach provides similarly robust privacy compliance as retraining all models, but significantly reduces the computational cost by only retraining models that have removed individuals. The infrastructure supports querying models by cohort. Automation can be used to scale out the system to handle the retraining of models affected removed individuals. Training and releasing updated models to end users can be done within a 30-90 day window (or other applicable window).

D. Surgical Removal from Reference Panel and Retraining of Classifiers

Because of the arduous nature of clustering, the initial design for privacy compliance may be to maintain the existing clustering data (minus the deleted individuals) and simply retrain the classifiers. In this scenario, individual level data is deleted from the reference panel responsive to a deletion request. The individual level data is removed and the classifiers are then retrained. Automation can handle training and validation. Retraining the classifiers can mitigate decay of classifier performance over time. Automation can be used for automatic promotion of models with retrained classifiers. A validation step can be used to monitor model performance decay over time. Depending on model performance and the number of individuals that are removed, the model can be retrained from scratch at a certain cadence or if certain performance changes are determined. Automation can be used to scale out the system to handle the retraining of models affected by privacy requests. Training and releasing updated models to end users can be done within a 30-90 day window (or other applicable window). Automatically promoted models would be used for new end users while older end users results would likely not need to be recomputed. Automated rules around validation can be used to evaluate the impact of deletions on model performance.

### VII. Metadata User Interface

It is desirable to have an automated way of taking a trained model from MLFlow and releasing it to production with minimal manual intervention or need for cross-team coordination. Automation can include the following features: extract trained model from MLFlow, edit user facing text, edit maps (overlays, colors, shapes, filesize, handles S3), Nesting/inheritance, versioning, changelog, version diffing, metadata import/export between environments, live data validation, decoupling data updates from service releases, model promotion/rollout controls. Some of these features are described in more detail below.

A. Extracting Trained Model from MLFlow

To do this, one finds the trained model to promote in MLFlow and clicks the promote button. Once that happens, the metadata UI can see the model and will pull in the relevant artifacts to create the top-level model metadata and cluster metadata.

Model metadata is mainly the model name and version and any defaults that will be used by any cluster that does not override the defaults.

Cluster metadata includes various user facing cluster labels, description text, cluster colors, text colors, demonyms, references to an existing ancestry population, a parent cluster if the cluster is part of a hierarchy, and GeoJSON defining the location of the cluster. Most of this data will not be known ahead of time and may need to be manually entered or altered.

The GeoJSON goes through multiple steps during the initial import. There are multiple versions of the GeoJSON per cluster based on predefined hyperparameters. The system can select the first one found, but can be manually overwritten if an alternative makes more sense. This will eventually become some kind of selector per cluster. Then, the shape's borders are intersected with existing ancestry maps to trim anything that falls outside of the existing maps. Next, the precision of the latitude/longitude are reduced to 5 decimal places (˜1 meter), as a first pass at reducing the total file size. Then a stable representation of the GeoJSON is used to compute a checksum. This checksum then becomes the GeoJSON filename in S3. Once checksum is known, the stable representation of the GeoJSON is uploaded to S3 with the checksum as the filename. The path to the GeoJSON is recorded and served through cloudfront.

Once the model is imported from MLFlow, it is ready for further processing.

B. Edit End-User Facing Text

Anyone who has access to the metadata tool should be able to quickly modify any of the end-user facing data without worrying about losing the data or entering invalid/incorrectly formatted data. This data was mostly being maintained in separate spreadsheets before, so the UI attempts to recreate the look and feel of spreadsheets for a lot of the metadata. The data input widget changes depending on the type of data being edited to help prevent typos or other mistakes.

The Ancestry Composition population of a given model or cluster is associated with a dropdown that is populated with all of the valid populations. If no population is appropriate for a model or cluster, this value can be left empty.

The cluster color and text color allow a hexadecimal specifier to be chosen and shows a preview of the color once entered. If the cluster is showing on a map, then the cluster will immediately be updated to the entered color.

The parent cluster, which allows definition of cluster hierarchies, is a dropdown populated by all valid clusters with which a cluster could be associated. A cluster cannot have itself as a parent so that is removed. A cluster also cannot have a parent that introduces a cycle, so any clusters that can introduce a cycle are removed. A cluster does not need to have a parent cluster, so an empty parent is a valid option too.

The labels and demonyms used to describe clusters can be text without too many rules. It can be plain text with no markup.

The description allows a small subset of text formatting markup (e.g., bold, italics, underline) and shows a preview of the formatted text when done editing the text.

The GeoJSON is not editable inside the spreadsheet, but it shows the expected file size of the GeoJSON. Editing the GeoJSON can be done in the map editor.

C. Editing GeoJSON

A map can be displayed below the spreadsheet editor or in some other location. The spreadsheet can have a checkbox that allows toggling a cluster on/off on the map. If the user only has one cluster selected, then the map editor options can appear allowing a user to modify the cluster GeoJSON. The editor is mostly powered by leaflet and geoman with some custom behaviors.

If a user wants to draw a new shape onto a polygon there are three polygon drawing tools, for a rectangle, a freeform polygon, or a circle. Once a user draws one of these shapes, the existing polygons of the GeoJSON are examined and a union is performed between them, so it all becomes one shape. Any drawn circles are converted into 64-sided polygons so they can be merged into the existing polygon.

If a user wants to change a few points on an existing polygon, there is a point editor tool. This will allow a user to take any point associated with a polygon and drag it to wherever they want. If the points are moved in a way that causes polygons to overlap, then the polygons will automatically be unioned together. To remove polygons or sections of polygons, the user can cut a freeform polygon. This uses the base geoman implementation with no customization.

In addition to the drawing and cutting tools, there are a few more editor customizations. Some of the GeoJSON associated with clusters can be very large in terms of file size (megabytes). It is desirable to keep response sizes relatively small, so these sizes should be reduced from time to time without losing the basic shape. To that end, a simplification tool that works based on the Visvalingam-Whyatt algorithm was added. It allows users to set the number of points to use in the GeoJSON and allows them to interactively find the ideal number of points. The shape will change live and the user can visually determine if an important point was removed. If the point is important, they can increase the number of points associated with the GeoJSON and it will immediately come back.

Another tool can view the raw GeoJSON inline. This allows quickly overwriting a cluster's GeoJSON with the exact shape wanted. This becomes important as users iterate models that have had their shapes manually modified.

All of these map edits are destructive to the underlying GeoJSON. Thus, hooks were added to an undo/redo stack so the user can quickly step through all of the edits that have been made to a cluster. If a change is not wanted, it can be undone and reverted to the exact version desired. This also allows for very quick comparisons between edits.

The clusters need various map contexts to help make sure the GeoJSON looks correct and that the text associated with the clusters is accurate with respect to what the map is displaying. There is a base world map with geopolitical boundaries and labels. Then there is the map shown in Ancestry Composition on top of that map. There is an option to swap in and out various other maps that can help create the best maps and region information.

D. Nesting/Inheritance

Model clusters typically connect to other population hierarchies defined by other systems, and these clusters also connect to other clusters. It is desirable to easily show these relationships and propagate the data to the various child clusters without needing to manually copy and paste to all of the relevant clusters.

There is the parent Ancestry Composition population. Since recent ancestor clusters (RAC) do not have to follow the exact same hierarchy defined by Ancestry Composition, it is possible for multiple clusters in the same model to have different connected Ancestry Composition populations. There can also be no connected population. The Ancestry Composition population can be defined at the model level and all clusters can inherit that population. Clusters can choose to override that population if needed. If no model level population is defined, then individual clusters might not be associated with a population unless specific clusters are associated with ancestry populations. No other inheritance exists at this level.

A RAC model can have clusters with sub-clusters, and this pattern can continue to an arbitrary number of levels. When a cluster is nested under another cluster, there will usually be common data to carry over without requiring full redefinition. A cluster can inherit from a first parent cluster that defines the data that is missing. If no parent cluster defines the data, the model may define the missing data. This evaluation is handled automatically by the API and in the metadata UI so that there is a consistent inheritance/nesting implementation across all clients and the scope of copy/paste errors/typos is reduced.

E. Versioning/Changelog

When editing the metadata associated with a model or cluster, it is desirable to always be able to see what the state of the model was at a given point in time, and to see how the model changed over time. To support this, on save the old metadata pointers are soft-deleted and updated to point to the new data location. Timestamps are recorded for when the edits were created. This allows recordkeeping for all changes, the dates the changes were made and who made the changes. This history combined with version diffing in the UI to allow tracking of how things have evolved over time, or to restore an old version.

F. Version Diffing

When a new version is created, it is desirable to see what has changed, but the way a change to text versus a change to a polygon is shown can be different. To highlight that a diff exists, the color of the cell in which the data is displayed is changed. To see the change, the user clicks into the cell and interacts with the undo/redo buttons. Since the GeoJSON is not shown in the grid, the file size cell will change color and the diff will be displayed in the map editor. The user can toggle between the shape diff or the raw GeoJSON to see what changed in the GeoJSON.

There are distinctions between saved versions and unsaved user edits. The user can swap out the underlying base revision and diff revision, and their unsaved edits will be replayed on top of those revision changes.

G. Metadata Import/Export

It is desirable to have the ability to easily move metadata around various environments so that users can test and debug issues with the metadata or develop new features against realistic production metadata. A copy to cUrl button allows exporting an API request that can be run against any environment, and it will recreate the exact state of the metadata at that point in time.

H. Live Data Validation

There are many ways in which data can be incorrect, and incorrect data can break client applications in various ways. There are multiple checks built into the data editors to prevent various types of invalid edits from being possible. If a user enters an invalid hex color it is rejected. If a user tries to create a cluster inheritance cycle, it is rejected. If a user enters invalid GeoJSON, the map will fail to render and it is rejected. If the GeoJSON is almost correct, operations can be performed to correct the data for the user and allow it.

### I. Decoupling Metadata Updates from Service Releases

The process of updating the service to address potential defects may include: creating a fix, pushing a git pull request, having the pull request reviewed and approved by a software developer, having the pull request verified by Cl/CD pipelines, merging the request into the main line of code, deploying to integration, and deploying to production. If everything goes well, this process can be completed in just under an hour. If updating the service involves updating the user facing product/website, this process will likely take about a day. If mobile clients are updated or coordination with multiple teams across multiple repositories is needed, the release can take days to weeks.

The process of updating the metadata to address potential defects may include: logging in as an administrator, fixing the metadata, and saving the metadata. If everything goes well, this process can be completed in under a minute.

It is desirable to define as much of the product as possible using metadata so that it can be decoupled from the slower release processes of code fixes, while also putting safeguards and audit logs in place to make it easy to revert changes.

J. Model Promotion/Rollout Controls

The initial promotion can happen with a button click in MLFlow. After that has occurred, there is another layer of rollout controls exposed by the RACmachine metadata UI. The stage can be changed from None to Staging to Production to Archived. None means the model is only visible to the metadata UI. Staging means the model should only be visible to testers. Live means the model should be live to actual users, but the percent of users that can see the feature can be controlled with a feature switch. Archived means the model is removed from any user facing interactions. All of these controls make it so that a model can be turned off and on in seconds, and there is control over who has access to it and when.

### VIII. Example Operations

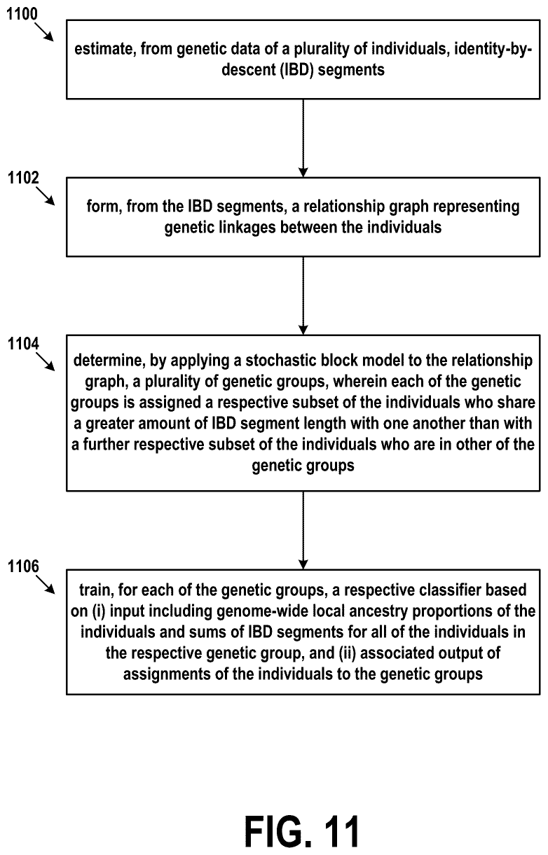

FIGS. 11-13 are flow charts illustrating example embodiments. The processes illustrated by FIGS. 11-13 may be carried out by a computing device, such as computing device 100, a cluster of computing devices, such as server cluster 200, and/or a genetic computing platform. However, the process can be carried out by other types of devices or device subsystems. For example, the process could be carried out by one or more commercial cloud-based computing platforms such as AWS.

The embodiments of FIGS. 11-13 may be simplified by the removal of any one or more of the features shown therein. Further, these embodiments may be combined with one another, as well as features, aspects, and/or implementations of any of the previous figures or otherwise described herein.

Block 1100 of FIG. 11 may involve estimating, from genetic data of a plurality of individuals, IBD segments.

Block 1102 may involve forming, from the IBD segments, a relationship graph representing genetic linkages between the individuals.

Block 1104 may involve determining, by applying a stochastic block model to the relationship graph, a plurality of genetic groups, wherein each of the genetic groups is assigned a respective subset of the individuals who share a greater amount of IBD segment length with one another than with a further respective subset of the individuals who are in other of the genetic groups.

Block 1106 may involve training, for each of the genetic groups, a respective classifier based on (i) input including genome-wide local ancestry proportions of the individuals and sums of IBD segments for all of the individuals in the respective genetic group, and (ii) associated output of assignments of the individuals to the genetic groups.

In some embodiments, estimating the IBD segments comprises determining segments of the genetic data of at least a threshold length and that are shared by two or more of the individuals who have a common ancestor.

In some embodiments, the individuals are represented by vertices in the relationship graph, and wherein the genetic linkages are represented by edges in the relationship graph.

In some embodiments, the genetic linkages are based on amounts of IBD segment length in common between pairs of the individuals.

In some embodiments, the assignments of the individuals to the genetic groups given the relationship graph is based on Bayesian inference from: (i) a calculated probability of observing the relationship graph given the assignments of the individuals to the genetic groups, (ii) a calculated probability of the assignments of the individuals to the genetic groups, and (iii) a sampled probability of the relationship graph.

In some embodiments, determining the plurality of genetic groups comprises: determining that a particular genetic group of the genetic groups contains less than a threshold number of individuals; and removing the particular genetic group from the plurality of genetic groups.

In some embodiments, the IBD segments for all of the individuals in the respective genetic group are merged so that any genetic regions with overlapping IBD segments are counted once.

Some embodiments may further involve: receiving further genetic data of a further individual; determining, from the further genetic data, a further genome-wide local ancestry proportion of the further individual; applying each of the respective classifiers to: (i) the further genome-wide local ancestry proportion of the further individual, and (ii) the sums of IBD segments for all of the individuals in the corresponding genetic group; and based on results of applying each of the respective classifiers, determining at least one genetic group to which the further individual belongs.

In some embodiments, the respective classifiers are deployed for production use. These embodiments may involve: receiving, on behalf of a particular individual of the plurality of individuals, a deletion request; in response to receiving the deletion request: (i) deleting, from the genetic data, particular genetic data of the particular individual, (ii) determining a set of one or more of the respective classifiers that were trained using at least some of the particular genetic data, and (iii) retraining the set of one or more of the respective classifiers with the genetic data after deletion; and redeploying the set of one or more of the respective classifiers as retrained.

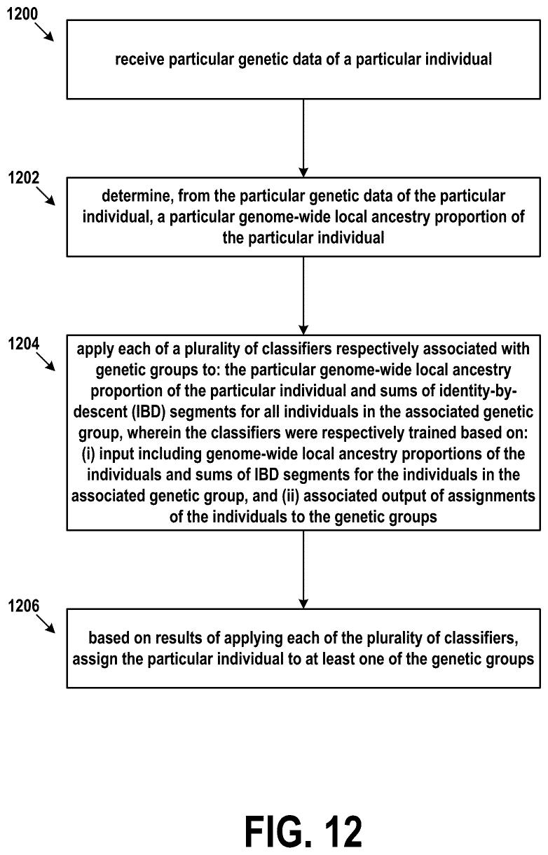

Block 1200 of FIG. 12 may involve receiving particular genetic data of a particular individual.

Block 1202 may involve determining, from the particular genetic data of the particular individual, a particular genome-wide local ancestry proportion of the particular individual.

Block 1204 may involve applying each of a plurality of classifiers respectively associated with genetic groups to: the particular genome-wide local ancestry proportion of the particular individual and sums of IBD segments for all individuals in the associated genetic group, wherein the classifiers were respectively trained based on: (i) input including genome-wide local ancestry proportions of the individuals and sums of IBD segments for the individuals in the associated genetic group, and (ii) associated output of assignments of the individuals to the genetic groups.

Block 1206 may involve, based on results of applying each of the plurality of classifiers, assigning the particular individual to at least one of the genetic groups.

In some embodiments, the IBD segments are of genetic data of at least a threshold length and that are shared by two or more of the individuals who have a common ancestor.

In some embodiments, the assignments of the individuals to the genetic groups is based on Bayesian inference from: (i) a calculated probability of observing a relationship graph given the assignments of the individuals to the genetic groups, (ii) a calculated probability of the assignments of the individuals to the genetic groups, and (iii) a sampled probability of the relationship graph.

In some embodiments, the genetic groups each contain at least a threshold number of individuals.

In some embodiments, the IBD segments for all of the individuals in each of the genetic groups are merged so that any genetic regions with overlapping IBD segments are counted once.

In some embodiments, the classifiers are based on logistic regression.

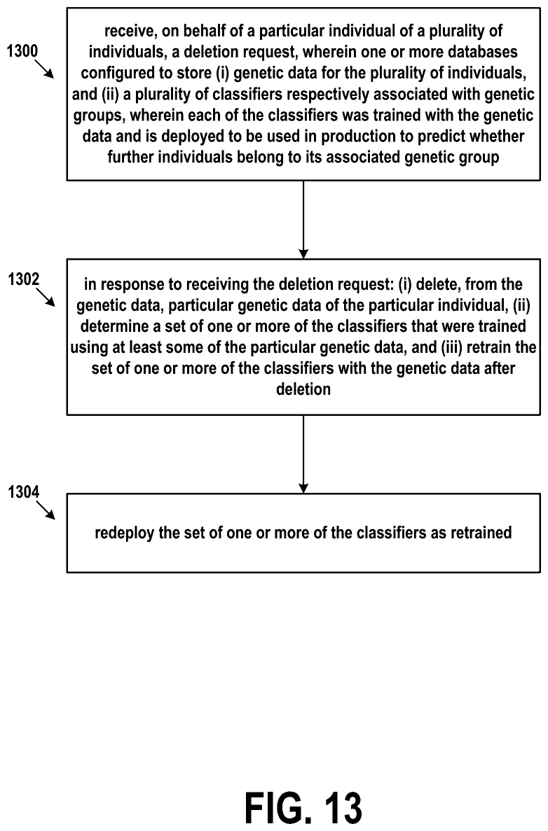

Block 1300 of FIG. 13 may involve receiving, on behalf of a particular individual of a plurality of individuals, a deletion request, wherein one or more databases configured to store (i) genetic data for the plurality of individuals, and (ii) a plurality of classifiers respectively associated with genetic groups, wherein each of the classifiers was trained with the genetic data and is deployed to be used in production to predict whether further individuals belong to its associated genetic group.

Block 1302 may involve, in response to receiving the deletion request: (i) deleting, from the genetic data, particular genetic data of the particular individual, (ii) determining a set of one or more of the classifiers that were trained using at least some of the particular genetic data, and (iii) retraining the set of one or more of the classifiers with the genetic data after deletion.

Block 1304 may involve redeploying the set of one or more of the classifiers as retrained.

In some embodiments, the one or more databases are also configured to store mappings between the individuals and their assigned genetic groups, wherein determining the set of one or more of the classifiers that were trained using at least some of the particular genetic data comprises: determining, from the mappings, that the particular individual has been assigned to a particular set of the genetic groups; and based on the particular set of the genetic groups, determining the set of one or more of the classifiers.

In some embodiments, retraining and redeploying the set of one or more of the classifiers is part of a periodic process, that occurs at a predetermined frequency, of retraining and redeploying all of the classifiers. In some embodiments, the predetermined frequency is between a 30-90 days, inclusive.

In some embodiments, retraining and redeploying the set of one or more of the classifiers is part of a periodic process, that occurs at a predetermined frequency, of retraining and redeploying only the classifiers impacted by deletions from the genetic data. In some embodiments, the predetermined frequency is between a 30-90 days, inclusive.

In some embodiments, the classifiers are based on logistic regression.

### IX. Closing

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step or block that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of computer readable medium such as a storage device including RAM, a disk drive, a solid-state drive, or another storage medium.

The computer readable medium can also include non-transitory computer readable media such as non-transitory computer readable media that store data for short periods of time like register memory and processor cache. The non-transitory computer readable media can further include non-transitory computer readable media that store program code and/or data for longer periods of time. Thus, the non-transitory computer readable media may include secondary or persistent long-term storage, like ROM, optical or magnetic disks, solid-state drives, or compact disc read only memory (CD-ROM), for example. The non-transitory computer readable media can also be any other volatile or non-volatile storage systems. A non-transitory computer readable medium can be considered a computer readable storage medium, for example, or a tangible storage device.

Moreover, a step or block that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments could include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

What is claimed is:

1. An article of manufacture including a non-transitory computer-readable medium, having stored thereon program instructions that, upon execution by a computing system, cause the computing system to perform operations comprising:
receiving particular genetic data of a particular individual;
determining, from the particular genetic data of the particular individual, a particular genome-wide local ancestry proportion of the particular individual;
applying each of a plurality of classifiers respectively associated with genetic groups to: the particular genome-wide local ancestry proportion of the particular individual and sums of identity-by-descent (IBD) segments for individuals in the associated genetic group, wherein the classifiers were respectively trained based on: (i) input including genome-wide local ancestry proportions of the individuals and sums of IBD segments for the individuals in the associated genetic group, and (ii) associated output of assignments of the individuals to the genetic groups; and
based on results of applying each of the plurality of classifiers, assigning the particular individual to at least one of the genetic groups.

2. The article of manufacture of claim 1, wherein the genetic groups were determined by:
estimating, from genetic data of a plurality of individuals, a plurality of IBD segments;
forming, from the plurality of IBD segments, a relationship graph representing genetic linkages between the individuals; and
determining, by applying a stochastic block model to the relationship graph, the genetic groups, wherein each of the genetic groups is assigned a respective subset of the individuals who share a greater amount of IBD segment length with one another than with a further respective subset of the individuals who are in other of the genetic groups.

3. The article of manufacture of claim 2, wherein the individuals are represented by vertices in the relationship graph, and wherein genetic linkages between the individuals are represented by edges in the relationship graph.

4. The article of manufacture of claim 3, wherein the genetic linkages are based on amounts of IBD segment length in common between pairs of the individuals.

5. The article of manufacture of claim 1, wherein the IBD segments are of genetic data of at least a threshold length and that are shared by two or more of the individuals who have a common ancestor.

6. The article of manufacture of claim 1, wherein the assignments of the individuals to the genetic groups is based on Bayesian inference from: (i) a calculated probability of observing a relationship graph given the assignments of the individuals to the genetic groups, (ii) a calculated probability of the assignments of the individuals to the genetic groups, and (iii) a sampled probability of the relationship graph.

7. The article of manufacture of claim 1, the operations further comprising:
receiving, on behalf of a further individual of the individuals, a deletion request;
in response to receiving the deletion request: (i) deleting, from genetic data of the individuals, further genetic data of the further individual, (ii) determining a set of one or more of the classifiers that were trained using at least some of the further genetic data, and (iii) retraining the set of one or more of the classifiers with the genetic data after deletion of the further genetic data; and
redeploying the set of one or more of the classifiers as retrained.

8. The article of manufacture of claim 1, wherein the IBD segments for the individuals in each of the genetic groups are merged so that any genetic regions with overlapping IBD segments are counted once.

9. The article of manufacture of claim 1, wherein the classifiers are based on logistic regression.

10. An article of manufacture including a non-transitory computer-readable medium, having stored thereon program instructions that, upon execution by a computing system, cause the computing system to perform operations comprising:
estimating, from genetic data of a plurality of individuals, identity-by-descent (IBD) segments;
forming, from the IBD segments, a relationship graph representing genetic linkages between the individuals;
determining, by applying a stochastic block model to the relationship graph, a plurality of genetic groups, wherein each of the genetic groups is assigned a respective subset of the individuals who share a greater amount of IBD segment length with one another than with a further respective subset of the individuals who are in other of the genetic groups; and
training, for each of the genetic groups, a respective classifier based on (i) input including genome-wide local ancestry proportions of the individuals and sums of IBD segments for the individuals in the respective genetic group, and (ii) associated output of assignments of the individuals to the genetic groups.

11. The article of manufacture of claim 10, wherein estimating the IBD segments comprises determining segments of the genetic data of at least a threshold length and that are shared by two or more of the individuals who have a common ancestor.

12. The article of manufacture of claim 10, wherein the individuals are represented by vertices in the relationship graph, and wherein the genetic linkages are represented by edges in the relationship graph.

13. The article of manufacture of claim 10, wherein the genetic linkages are based on amounts of IBD segment length in common between pairs of the individuals.

14. The article of manufacture of claim 10, wherein the assignments of the individuals to the genetic groups given the relationship graph is based on Bayesian inference from: (i) a calculated probability of observing the relationship graph given the assignments of the individuals to the genetic groups, (ii) a calculated probability of the assignments of the individuals to the genetic groups, and (iii) a sampled probability of the relationship graph.

15. The article of manufacture of claim 10, wherein determining the plurality of genetic groups comprises:
determining that a particular genetic group of the genetic groups contains less than a threshold number of individuals; and
removing the particular genetic group from the plurality of genetic groups.

16. The article of manufacture of claim 10, wherein the IBD segments for the individuals in the respective genetic group are merged so that any genetic regions with overlapping IBD segments are counted once.

17. The article of manufacture of claim 10, the operations further comprising:
receiving further genetic data of a further individual;
determining, from the further genetic data, a further genome-wide local ancestry proportion of the further individual;
applying each of the respective classifiers to: (i) the further genome-wide local ancestry proportion of the further individual, and (ii) the sums of IBD segments for the individuals in the corresponding genetic group; and
based on results of applying each of the respective classifiers, determining at least one genetic group to which the further individual belongs.

18. The article of manufacture of claim 10, wherein the respective classifiers are deployed for production use, the operations further comprising:
receiving, on behalf of a particular individual of the plurality of individuals, a deletion request;
in response to receiving the deletion request: (i) deleting, from the genetic data, particular genetic data of the particular individual, (ii) determining a set of one or more of the respective classifiers that were trained using at least some of the particular genetic data, and (iii) retraining the set of one or more of the respective classifiers with the genetic data after deletion; and
redeploying the set of one or more of the respective classifiers as retrained.

19. The article of manufacture of claim 10, wherein the respective classifiers are based on logistic regression.

20. A genetic computing platform comprising:
one or more databases configured to store (i) genetic data for a plurality of individuals, and (ii) a plurality of classifiers respectively associated with genetic groups, wherein each of the classifiers was trained with the genetic data and is deployed to be used in production to predict whether further individuals belong to its associated genetic group; and
one or more processors configured to:
receive, on behalf of a particular individual of the plurality of individuals, a deletion request;
in response to receiving the deletion request: (i) delete, from the genetic data, particular genetic data of the particular individual, (ii) determine a set of one or more of the classifiers that were trained using at least some of the particular genetic data, and (iii) retrain the set of one or more of the classifiers with the genetic data after deletion; and
redeploy the set of one or more of the classifiers as retrained.
